(19) United States
(12) Patent Application Publication
(10) Pub. No.: US 2012/0122700 A1
(43) Pub. Date: May 17, 2012

(54) MATERIALS AND METHODS FOR DETERMINING SUBTELOMERE DNA SEQUENCE

(75) Inventors: Peter G. Medveczky, Lutz, FL (US); Maria M. Medveczky, Lutz, FL (US); Jesse H. Arbuckle, Tampa, FL (US)

(73) Assignee: University of South Florida, Tampa, FL (US)

(21) Appl. No.: 13/295,803

(22) Filed: Nov. 14, 2011

Related U.S. Application Data
(60) Provisional application No. 61/412,476, filed on Nov. 11, 2010.

(51) Int. Cl. C40B 20/00; C12Q 1/70; C07H 21/04

(52) U.S. Cl. 506/2; 536/24.33; 435/5

(57) ABSTRACT

The subject invention pertains to methods for rapid and accurate determination of subtelomere DNA sequences. Also provided are kits for determination of subtelomere sequences and uses of chromosomal terminal sequences for studying pathogenesis and treatment of diseases.

### CROSS-REFERENCE TO RELATED APPLICATION

This application claims the benefit of U.S. provisional application Ser. No. 61/412,476, filed Nov. 11, 2010, which is herein incorporated by reference in its entirety.

### GOVERNMENTAL SUPPORT

This invention was made with government support under Grant No. RO1CA111196 awarded by the National Institutes of Health (NIH). The government has certain rights in the invention.

### BACKGROUND OF THE INVENTION

A subtelomere, usually 300 to 500 kb in length, is a chromosomal region located immediately adjacent to the centromere side of the (5′TTAGGG3′)ₙ telomere repeat array.

Subtelomeres serve as the transition zone between chromosome-specific sequences and the telomere repeat arrays, and are typically composed of duplicated segments of perfect telomere repeat sequences interspersed with highly homologous imperfect telomere repeats and variable coding sequences. Studies have suggested that human subtelomeres are hot spots of dynamic interchromosomal recombination and segment duplication. While subtelomere DNA rearrangements lead to rapid gene innovation and contribute to phenotypic diversity among individuals, even a subtle mis-rearrangement could result in impairment or loss of important gene function. Many deleterious genetic disorders including mental retardation have been causally linked to displacement or deletion of subtelomere genes.

Although the initial draft sequence of the human genome leads to the identification of more than 1000 genes associated with different human diseases, full sequence coverage of the subtelomere region has not been achieved. The extensively duplicated subtelomere segments have complicated the mapping and sequencing efforts and caused a disproportionate number of errors in the initial draft. Currently, the subtelomere sequences of about half of the human chromosomes remain uncharacterized. These unveiled sequences are essential starting points for identification and analysis of subtelomere genes, which could lead to major breakthroughs in the diagnosis and therapy of a range of genetic diseases. Therefore, improved technology allowing for determination of unknown subtelomere sequences is urgently needed.

Human herpesvirus-6 (HHV-6), a betaherpesvirus related to the human cytomegalovirus, was first discovered in HIV-infected patients suffering from lymphoproliferative disorders (1). HHV-6 viruses can be categorized into two subspecies: HHV-6A and HHV-6B (2,3,5). Following primary infection, both subspecies remain in a persistent/latent state for the life of the host (7, 8), and may reactivate in immunocompetent and, more often, in immunosuppressed hosts. Of the two subspecies, HHV-6A is more neurovirulent as evidenced by increased concentration of viral DNA in the plaques of MS patients and its ability to establish latency in oligodendrocytes (5, 16, 17). HHV-6B infects over 90% of children and is the primary cause of exanthem subitum (4, 6). HHV-6 viruses, which have always been associated with their adverse health impacts, become beneficially used in the present invention for determining subtelomere sequences.

### BRIEF SUM MARY OF THE INVENTION

The subject invention provides methods for rapid and accurate determination of subtelomere DNA sequence. The subject invention also permits precise identification of the telomere sequence into which HHV-6 DNA is inserted in infected individuals.

In an embodiment, the method comprises:

a) providing a population of host cells that carry HHV-6 genomic DNA in a host chromosome;

b) subjecting host chromosomal DNA to inverse polymerase chain reaction (IPCR), thereby generating HHV-6-subtelomere DNA; and

c) determining the HHV-6-subtelomere DNA sequence.

In a further embodiment, if novel subtelomere sequence is identified, the method further comprises identifying the host chromosome(s) into which the subtelomere sequence is inserted. In an embodiment, host chromosome(s) into which the subtelomere sequence is inserted can be identified by in situ fluoreccent hybridization (FISH) using HHV-6-specific DNA probes.

In addition, the subject invention provides kits for determination of subtelomere sequences, comprising an IPCR primer of the subject invention useful for amplification of the subtelomere DNA.

In addition, the subject invention pertains to uses of subtelomere sequences for studying pathogenesis and treatment of diseases associated with subtelomere sequences. The subject invention can also be used for diagnosis and treatment of diseases associated with HHV-6 infection.

### BRIEF DESCRIPTION OF THE DRAWINGS

FIG. 1 shows that HHV-6 genomic DNA is present in the host genomic fraction of Family-1 T-cells. One million T-cells were isolated from Family-1 members, control uninfected PBMCs, and HHV-6 positive Katata cell line (HHV-6B integrated Burkitt's lymphoma cell line) (37). Cells were loaded on a vertical agarose gel and analyzed for episomal, linear, or integrated DNA by the method of Gardella et al. (21). (a) Southern hybridization results using HHV-6A probe (Left panel). Blot was stripped and hybridized with mitochondrion oligonucleotide probe (Right panel) (41). (b) T-cells of family members immortalized with H. saimiri strain C484 were subjected to Gardella gel analysis and hybridized with HHV-6 and H. saimiri probes.

FIG. 2 illustrates chromosome 17p subtelomere-specific PCR amplification of DNA of members of Family-2. (a) depicts the putative HHV-6-chromosome 17p-HHV-6A junction. Arrows indicate position of primers (drawing is not in scale) derived from the direct repeat left (DR_{L}) and right (DR_{R}) of the viral genome (Unique long=U_{L}) and to chromosome 17p subtelomere (28). (b) Genomic DNA of Family-2 Mother and Sibling and HHV-6A infected and uninfected Jjhan cells was amplified. PCR products were separated by electrophoresis and analyzed by Southern blotting using ³²P-labelled oligonucleotide probes as indicated at the bottom of each panel. Co-hybridization of a 1.5 kb fragment with all three probes suggests HHV-6A chromosome joining with viral DR_{R} telomere repeat. (c) The predominant 1.5 kb amplicon from Family-2/Mother was cloned (n=3) and sequenced. GenBank homology search confirmed the presence of chromosome subtelomere 17p sequence (top) joined with TTAGGG telomere repeats (bold and underlined), and HHV-6 DR_{R} sequence (bottom).

FIG. 3 shows that PCR amplification fails to detect HHV-6 DNA in episomal fractions of CsCl/ethidium bromide (EtBr) gradients. To search for covalently linked circular viral episomes by a method more sensitive than the method of Gardella, 50 μg DNA from two latently infected HEK-293 clones, T-cells from Family-2/Mother, and T-cells from Family-1/Sibling-2 immortalized with HVS strain C484 were subjected to CsCl/EtBr gradient ultracentrifugation for two days (CsCl density 1.55 g/ml, 10 μg/ml EtBr, VTI 65 rotor 40K rpm). After centrifugation, fractions were collected and linear and episomal (ccc) DNA was identified by agarose electrophoresis. Salt and EtBr were removed from combined linear and episomal ccc fractions and were subjected to PCR based amplification using primers to (a) HHV-6 ORF-U94 and mitochondrial cytochrome c oxidase (positive episomal control), (b) HHV-6 ORF-U94 and beta actin genomic positive control, and (c) HHV-6 ORF-U94, C484 Stp, and cytochrome c oxidase (positive episomal control). “ccc” stands for covalently closed circular episomal fraction.

FIG. 4 shows HHV-6 DNA qPCR analysis of patients' T-cells and in vitro latently infected HEK-293 cell lines induced by TPA and TSA. T-cell cultures from five family members and three latently infected HEK-293 cell lines were cultured in AIM-V or DMEM medium supplemented with 10% FCS and treated with known inducers of herpesvirus lytic replication protein kinase-C inducer TPA (20 ng/ml) and histone deacetylase inhibitor Trichostatin-A (TSA) (80 ng/ml) for three days (42, 43). DNA isolated from cells (in triplicate) were subjected to quantitative real time PCR (qPCR) for ORF U94 and fold change ratios of Ct values normalized to beta actin were relative to untreated control. (a) HEK-293 (n=3) and (b) T cells (n=5) (see FIG. 12). TSA promoted significant increase in viral DNA replication, while the stimulation with TPA and hydrocortisone had a milder effect. Statistical analysis was based upon the student T-test and p<0.05 was considered significant. (c) Gardella gel analysis of infected Molt3 cells two weeks after co-culturing with PBMCs treated with TPA from Family-1/Sibling-2. Hector 2 T-cell line carrying one copy of HHV-6A per a cell.

FIG. 5 shows EcoRI restriction site analysis of integrated HHV-6 in Family-1 T cells. Total genomic DNA was isolated from HHV-6 integrated Family-1 members' PBMCs, HHV-6A (U1102 strain) infected Jjhan cells, HHV-6B (Z29 strain) infected Molt3 cells, and uninfected PBMCs with Wizard Genomic DNA Purification Kit (Promega) according to manufacturer's protocol. 5 μg of genomic DNA was digested with 24 U of EcoRI (Promega) and separated on 0.8% horizontal agarose gel. Southern hybridization was accomplished with HHV-6A (U1102) cosmid (40) probe mixture.

FIG. 6A-H (continuous) shows multiple sequence alignment of ORF U94 and left repeat from chromosomally integrated and reactivated HHV-6. ORF U94 was amplified with primers U94L-1, U94L-2, U94R-1, and U94R-2; while the left repeat was amplified with primers LTR-1 and LTR-2 from genomic DNA isolated from chromosomally integrated family members. Alignments were completed with ClustalX 2.0 multiple sequence alignment software.

FIG. 7A-C shows micrographs of fluorescent in situ hybridization (FISH) of metaphase chromosomes from T-lymphocytes of family members. PBMCs were stimulated for 72 hours with 20 ng/ml phytohemagglutinin (PHA), and then cultured in RPMI 1640 medium containing 50 U/ml IL-2 and 10% FCS. Metaphase chromosomes were generated according to standard cytogenic protocol (Kowalska et al., Chromosome Research, 2007), stained with DAPI and hybridized with various probes as follows; (a) FITC-conjugated HHV-6A (U1102 strain) cosmid probes pMF311-2 and pMF335-6 (green) (40) and cy5-conjugated telomere peptide nucleic acid (PNA) probe (red) (DakoCytomation). The viral genome was present in the same chromosome of the corresponding parents and siblings, suggesting germ line transmission (26): Family-1 chromosome 18q23; Family-2 chromosome 17p13.3; and Family-3 chromosome 22q. Lower right hand corner of each figure contains magnification of the chromosome with integrated viral DNA. (b) Representative FISH from members of Families 1-3. Hybridization with FITC-conjugated HHV-6A (U1102 strain) cosmid probes pMF311-2 and pMF335-6 (green top row) (40). (c) cy5-conjugated BCL2 probe (Chromosome 18) (red), FITC-conjugated chromosome 17 centromere probe (green), and cy5 and FITC-conjugated EWSR1 probe (Chromosome 22) (dual labeled-red and green bottom row) (Vysis).

FIG. 8A-B (continuous) shows sequencing results of virus-chromosome junction from HHV-6A-infected Jjhan cells. DNA from HHV-6A (U1102) infected Jjhan cells were subjected to amplification by PCR primers derived from HHV-6A DR_{R} and subtelomere primers 11q, 17p and 18q published by Baird et al. (28). After cloning and sequencing of PCR products, data were subjected to GenBank nucleotide sequence analysis. Virus-chromosome-telomere repeat viral genome junctions are indicated by brackets and TTAGGG telomere repeats are indicated as underlined and in bold.

FIG. 9 shows microscopic images and fluorescent cell sorting of GFP expression by recombinant HHV-6A (HHV-6AGFP) in Jjhan cells. (a) Construction of recombinant HHV-6A (U1102) virus expressing GFP (HHV-6AGFP). HHV-6AGFP was constructed by inserting EGFP expression cassette cloned into pBeloBAC11 (New England BioLabs) between the two polyadenylation signals of ORF U53 and U54 (85,981-86,034 bp) through homologous recombination. HHV-6A strain U1102 DNA ORFU53 and U54 fragments were amplified with the following primers containing restriction sites BamHI and SacII: U53L and U53R primers; U54L and U54R. The pEGFP-N2 vector template (BD Biosciences Clontech) was used to amplify the GFP expression cassette driven by the human cytomegalovirus immediate early gene promoter with primers GFP-N2-L and GFP-N2-R. Sub-cloning of ORF U53, ORF U54, and GFP was completed in pBluescript SK+ (Invitrogen) followed by sequencing to confirm lack of PCR generated mutations. Fragments were then sequentially ligated between the restriction sites BamHI and HindIII of pBeloBac11 with T4 DNA ligase (Promega). To allow homologues recombination, BAC/GFP vector with ORF U53 and U54 was linearized with SacII. (b) Infection of Jjhan cells resulted in an increased number of green fluorescent cells from 7 to 37 days post-infection as shown by fluorescent microscopy (100× magnification). (c) Flow cytometric analysis indicated that 60% of Jjhan cells express GFP after 37 days post-infection with HHV-6AGFP. (d) DNA from HHV-6AGFP infected Jjhan cells, uninfected Jjhan and Molt3 cells, and HHV-6A infected Jjhan cells was isolated. PCR amplified ORF U53 with primers U53L and U53R produced a 2033 bp amplicon in HHV-6A infected cells.

FIG. 10 shows detection of the viral genome by PCR and fluorescent in situ hybridization (FISH) of HEK-293 cell clones latently infected with HHV-6A (U1102). (a) Genomic DNA was isolated from HEK-293 cell clones and HHV-6A amplification was amplified with primers U94L-2 and U94R-1. A 215 bp fragment representing ORF U94 was observed in five HEK-293 clones. (b) Representative FISH from two independent HEK-293 cell clones of each of three HEK-293 clones. Hybridization was performed with HHV-6 cosmids pMF311-12 and pMF335-631 labeled with Fluorescein (green) (40). HEK-293/clone-1 had two independent chromosome-associated HHV-6A signals, while HEK-293/clone-2 and HEK-293/clone-3 only had one. HHV-6-hybridizing chromosome labeled with white arrow and higher magnification of the chromosome shown in red box. Metaphase chromosomes were stained with DAPI.

FIG. 11 illustrates inverse PCR (IPCR) (30) analysis of DNA from HHV-6A infected HEK-293 clones and from Family-1. (a) shows that integration of HHV-6 occurs within the telomere of a human chromosome via homologous recombination with the TTAGGG perfect repeats within Direct Repeat right (DR_{R}). IPCR involves digestion of genomic DNA with MboI followed by self-circularization of HHV-6 DR_{R} and adjacent chromosomal fragment. PCR amplification of HHV-6-chromosome fragment is achieved through HHV-6 primers IPCR-1 and IPCR-2. Co-hybridization of IPCR probe and telomere probe via Southern hybridization tests integration into human telomeres. DR_{L}=Direct Repeat left, U_{L}=Unique Long. (b) HEK-293 clones 1, 2, 3, 4 DNA were digested with MboI, diluted to prevent ligation of unrelated fragments, ligated and amplified. IPCR products were analyzed by Southern blot hybridization with telomere oligonucleotide probe (left panel), and with HHV-6 oligonucleotide probe-1 (right panel). Control IPCR was performed using DNA without ligation (unligated). (c) Southern hybridization of IPCR DNA products from members of Family-1. Telomere probe=left panel, HHV-6 Probe (IPCR-probe)=right panel.

FIG. 12 shows stimulation of HHV-6A DNA replication in individual T-cell cultures by qPCR analysis. Individual family members' T-cells were cultured in the presence of TPA (20 ng/ml) and trichostatin A (TSA, 80 ng/ml) for three days to promote virus reactivation. DNA isolated from cells (in triplicate) was subjected to quantitative real time PCR (qPCR) for ORF U94 and fold change ratios of Cq values normalized to beta actin were depicted relative to untreated control.

FIG. 13 shows reactivation of CI-HHV-6 induces syncytium formation as well as linear viral DNA and RNA. (a-d) Freshly isolated PBMC from Family-1, Sibling-2 stimulated with 20 ng/ml TPA and 1×10⁻⁶ M hydrocortisone. PBMC were cocultured with Molt3 cells for 2 weeks. Syncytium formation (400× magnification) was documented. (e) Molt3 cells treated with TPA and hydrocortisone and (f) Molt3 cells untreated. (g) Gardella gel analysis of Molt3 cells two weeks after coculturing with PBMC (center and right panel lane Molt3) and control uninduced T cell DNA (left panel and center and right panels “Hector 2” cell line carrying onecopy per cell HHV6A).

FIG. 14 shows that DNA binding (DB) and endonuclease active site (ENDO) of AAV-5 is conserved in U94/rep of HHV-6 (underlined).

FIG. 15 illustrates plasmid and viral constructs useful according to the subject invention. Left panel: plasmid constructs. pNeo-Tel encodes a selectable marker Neo/G14, two blocks of telomere repeats and a unique KpnI site to linearize the plasmid. Right panel: GFP/BAC recombinant HHV-6A was generated as described (Arbuckle et al., 2010) and will be used to clone the viral genome in E. coli.

FIG. 16 shows possible TERRA transcripts. Solid filled box=subtelomere; grey shaded boxes=telomeres; open box=HHV-6A genome. TERRA expression in normal chromosomes (top) or HHV-6A integrated chromosome (bottom). Primer codes; FS=forward subtelomere, RT=reverse telomere, FH=forward herpes, RH=reverse herpes.

FIG. 17 shows interaction of telomeres (horizontal lines) with TERRA (dashed line) and shelterin proteins (as indicated by abbreviations).

FIG. 18 shows potential impact of HHV-6A integration on telomere length. Insertion of the viral genome into TTAGGG telomere repeats may not significantly alter overall telomere length (top model). However, integration of HHV-6 genome near the end or center may change the overall telomere length.

FIG. 19 illustrates the STELA assay. DNA is depicted as boxes, and TTAGGG repeats are vertically patterned. DNA isolated from cells carrying HHV-6A in one chromosome allele is ligated with the “telorette” primer. Next, telomeres from the normal chromosome are amplified using the subtelomere and telltail primers. Telomeres of the chromosome allele containing integrated HHV-6A are amplified using HHV-6A left-end and telltale primers. PCR products are analyzed by Southern blotting using probes derived from subtelomere or HHV-6A sequences.

FIG. 20 shows that homologous recombination (HR) between two chromosomes mediated by the U94/rep complex could drastically shorten telomere length of a chromosome. Specifically, the chromosome depicted on the bottom is expected to undergo rapid loss of telomeres.

### BRIEF DESCRIPTION OF THE SEQUENCES

SEQ ID NO: 1 is a nucleic acid sequence of an IPCR primer useful according to the subject invention.

SEQ ID NO: 2 is a nucleic acid sequence of an IPCR primer useful according to the subject invention.

SEQ ID NO: 3 is a nucleic acid sequence of an IPCR probe useful according to the subject invention.

SEQ ID NO: 4 is a nucleic acid sequence of a telomere repeat probe useful according to the subject invention.

SEQ ID NO: 5 is a nucleic acid sequence derived from HHV-6 DR_{R}.

SEQ ID NO: 6 is a nucleic acid sequence derived from HHV-6 DR_{L}.

SEQ ID NO: 7 is a nucleic acid sequence of a chromosomal-specific PCR primer useful according to the subject invention.

SEQ ID NO: 8 is a nucleic acid sequence of a chromosomal-specific PCR primer useful according to the subject invention.

SEQ ID NO: 9 is a nucleic acid sequence of a chromosomal-specific PCR primer useful according to the subject invention.

SEQ ID NO: 10 is a nucleic acid sequence of a HHV-6 DR_{R} probe useful according to the subject invention.

SEQ ID NO: 11 is a nucleic acid sequence of a chromosomal-specific probe useful according to the subject invention.

SEQ ID NO: 12 is a nucleic acid sequence derived from HHV-6 DR₋₁.

SEQ ID NO: 13 is a nucleic acid sequence derived from HHV-6 DR₋₂.

SEQ ID NO: 14 is a nucleic acid sequence derived from HHV-6 U94 open reading frame.

SEQ ID NO: 15 is a nucleic acid sequence derived from HHV-6 U94 open reading frame.

SEQ ID NO: 16 is a nucleic acid sequence derived from HHV-6 U94 open reading frame.

SEQ ID NO: 17 is a nucleic acid sequence derived from HHV-6 U94 open reading frame.

SEQ ID NO: 18 is a nucleic acid sequence derived from a mitochondrion DNA of an HHV-6-infected individual.

SEQ ID NO: 19 is a nucleic acid sequence useful according to the subject invention.

SEQ ID NO: 20 is a nucleic acid sequence useful according to the subject invention.

SEQ ID NO: 21 is a nucleic acid sequence useful according to the subject invention.

SEQ ID NO: 22 is a nucleic acid sequence useful according to the subject invention.

SEQ ID NO: 23 is a nucleic acid sequence useful according to the subject invention.

SEQ ID NO: 24 is a nucleic acid sequence useful according to the subject invention.

SEQ ID NO: 25 is a nucleic acid sequence useful according to the subject invention.

SEQ ID NO: 26 is a nucleic acid sequence useful according to the subject invention.

SEQ ID NO: 27 is a nucleic acid sequence of a PCR primer useful according to the subject invention.

SEQ ID NO: 28 is a nucleic acid sequence of a PCR primer useful according to the subject invention.

SEQ ID NO: 29 is a nucleic acid sequence of a PCR primer useful according to the subject invention.

SEQ ID NO: 30 is a nucleic acid sequence of a PCR primer useful according to the subject invention.

SEQ ID NO: 31 is a nucleic acid sequence of a PCR primer useful according to the subject invention.

SEQ ID NO: 32 is a nucleic acid sequence of a PCR primer useful according to the subject invention.

SEQ ID NO: 33 is a nucleic acid sequence useful according to the subject invention.

SEQ ID NO: 34 is a nucleic acid sequence useful according to the subject invention.

SEQ ID NO: 35 is a nucleic acid sequence useful according to the subject invention.

SEQ ID NO: 36 is a nucleic acid sequence useful according to the subject invention.

SEQ ID NO: 37 is a nucleic acid sequence useful according to the subject invention.

SEQ ID NO: 38 is a nucleic acid sequence useful according to the subject invention.

SEQ ID NO: 39 is a nucleic acid sequence of a U94 PCR primer useful according to the subject invention.

SEQ ID NO: 40 is a nucleic acid sequence of a U94 PCR primer useful according to the subject invention.

### DETAILED DISCLOSURE OF THE INVENTION

The subject invention provides IPCR-based methods for rapid and accurate determination of a subtelomere DNA sequence. In addition, the subject invention permits identification of the telomere sequence into which HHV-6 DNA is inserted in infected individuals. Also provided are kits for determination of subtelomere sequences and/or telomere site of HHV-6 integration as well as uses of subtelomere/telomere sequences for studying pathogenesis and treatment of diseases.

Previous researchers have observed that individuals infected with HHV-6 viruses exhibited characteristically persistent and abnormally high levels of viral DNA in serum, plasma and whole blood. In addition, the presence of high levels of viral DNA has been frequently observed among infected family members, such as between parent-child or among siblings, indicating that HHV-6 viral DNA may be vertically transmitted via the germline (19-25). To explain these occasional observations, previous studies have used fluorescence in situ hybridization (FISH) to investigate whether HHV-6 genome DNA is integrated into the chromosomes of host cells. One significant disadvantage of FISH, however, is its lack of ability to distinguish non-covalent linkage of episomal DNA to the chromosome from chromosomal integration of viral DNA. As a result, although FISH results showed spots of illumination emitted from labeled HHV-6 DNA in the telomeric region, these illuminated spots could be due to the formation of non-covalent linkages between host chromosomes and viral episomes, rather than the integration of viral DNA into the host chromosomes.

It has now been discovered that the integration of viral DNA into the host chromosomes is the sole mechanism through which HHV-6 achieves latency during infection. As is demonstrated in Examples 2-6, HHV6-A integrates into the telomeres of human peripheral mononuclear cells in vivo and in vitro (such as in Jjhan T-cells and HEK-293 cells), and no viral episomal DNA is detected even using highly sensitive PCR assays. In cell lines capable of supporting productive, lytic infection, some of the cells quickly become latently infected by HHV-6 through chromosomal integration and remain viable. In addition, the latent, integrated HHV-6 genome is inducible after TPA or TSA stimulation. Co-cultivation studies also indicated that the latent, integrated genome was capable of producing fully competent virus.

The subject invention also involves the discovery that HHV-6 DNA integrates into the host genome via homologous recombination with human telomeres. The HHV-6A genome encodes a perfect TTAGGG telomere repeat array at the right end direct repeat (DR_{R}) and an imperfect TTAGGG repeat at the left end direct repeat (DR_{L}). The perfect TTAGGG repeats encoded in the right and left direct repeats of the viral genome mirror the human telomeric repeats. It is thus postulated that the left end of the viral genome is joined with a long array of TTAGGG repeats.

### Determination of Subtelomere DNA Sequence

One aspect of the subject invention provides methods for rapid and accurate determination of a subtelomere DNA sequence. In an embodiment, the method comprises:

a) providing a population of host cells that carry HHV-6 genomic DNA in host chromosomal DNA;

b) subjecting host chromosomal DNA to inverse polymerase chain reaction (IPCR), thereby generating HHV-6-subtelomere DNA; and

c) determining the HHV-6-subtelomere DNA sequence.

In a further embodiment, if a novel subtelomere sequence is identified, the method further comprises identifying the host chromosome(s) into which the subtelomere sequence is inserted. In an embodiment, host chromosome(s) into which the subtelomere sequence is inserted can be identified by in situ fluoreccent hybridization (FISH) using HHV-6-specific DNA probes. Alternatively, the method of the subject invention can be used to determine a previously unknown subtelomere sequence of a particular host chromosome of interest.

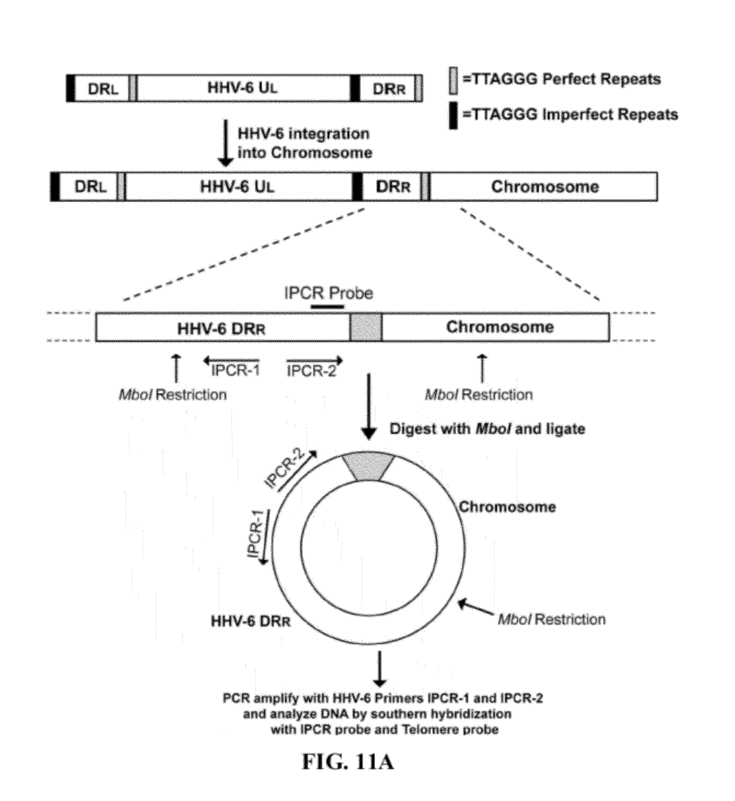

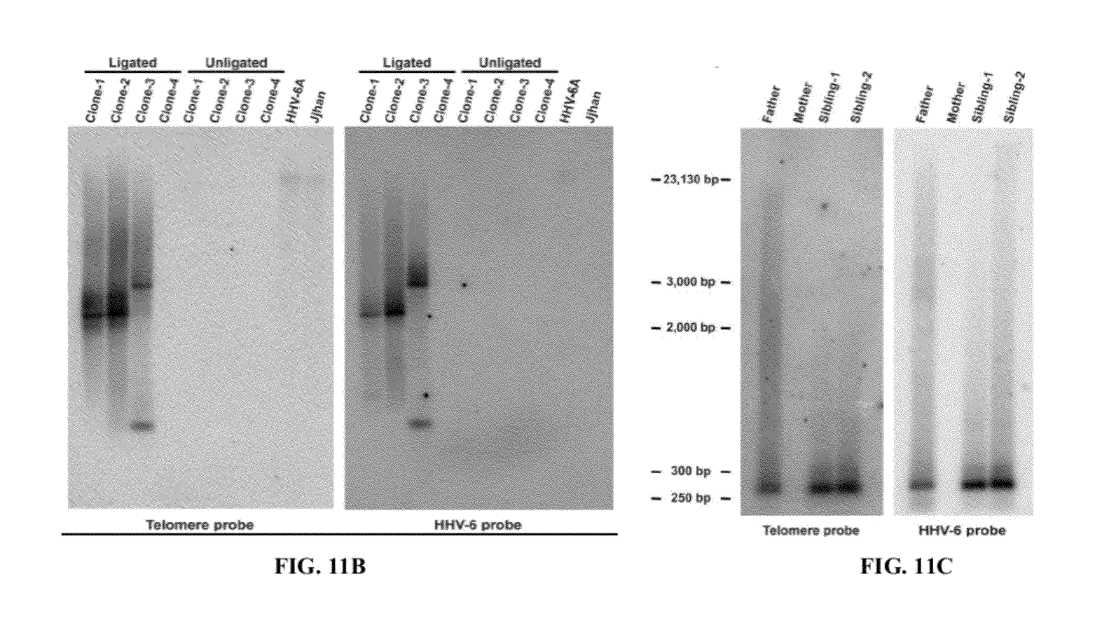

In one embodiment, the method for determining a subtelomere DNA sequence according to the subject invention is illustrated in FIG. 11 (see Example 1).

Host cells carrying HHV-6 genomic DNA in telomeric regions can be prepared by isolating cells from HHV-6 infected individuals. For instance, peripheral blood mononuclear cells (PMBC) can be derived from peripheral blood samples of HHV-6 infected individuals. Additionally or alternatively, cells carrying HHV-6 genomic DNA in telomeric regions can be prepared by infecting naïve cells with HHV-6 viruses or viral DNA, such as transfecting naïve cells with a DNA vector carrying HHV-6 viral DNA.

Host cells carrying HHV-6 genomic DNA in telomeric regions can be, for example, blood cells such as B lymphocytes, T lymphocytes, leukocytes, erythrocytes, macrophages, neutrophils, and umbilical cord blood stem cells; epithelial cells; connective tissue cells such as fibroblasts; neuronal cells such as astrocytes; kidney cells; pancreatic cells; and liver cells. In specific embodiments, human T cells Jjhan, Molt3 cells, Burkitt's lymphoma cells Raji, and/or human embryonic kidney-293 cells (HEK-293) are used for in vitro integration of HHV-6 genomic DNA into host chromosomes. Preferably, host cells are of mammalian origin, more preferably, of human origin.

Host chromosomes (e.g., mammalian or human chromosomes) that carry HHV-6 genomic DNA can be identified using fluorescence in situ hybridization (FISH), chromosome-specific PCR, and hybridization using chromosome-specific nucleic acid probes. Probes of the subject invention may be labeled to facilitate visualization and identification of target DNA. Labels include, for example, chromophores, fluorophores, dyes, phosphorescent groups, and radioactive materials (e.g., ³²P-labeled radioactive probes). Examples 2-4 illustrate certain embodiments of the subject invention for determining the specific host chromosomes into which HHV-6 genomic DNA is inserted.

Probes of the subject invention include oligonucleotides designed to hybridize specifically with target nucleic acids (e.g., HHV-6 DNA, telomere DNA, and subtelomere DNA). For instance, a telomeric probe can be an oligonucleotide comprising a sequence that hybridizes to a sequence contained within telomere repeats. As human telomeres comprise repeats of sequence 5′-TTAGGG-3′, a human telomere probe can comprise a sequence such as 5′-CCCTAA-3′ (for an RNA probe, 5′-CCCUAA-3′) or 5′-CTAACC-3′. Typically, an oligonucleotide probe will be 8 or more nucleotides in length, preferably 12, 15, 20 or more nucleotides in length.

“Hybridization” refers to a reaction in which one or more polynucleotides react to form a complex that is stabilized via hydrogen bonding between a particular purine and a particular pyrimidine in double-stranded nucleic acid molecules (DNA-DNA, DNA-RNA, or RNA-RNA). The major specific pairings are guanine with cytosine and adenine with thymine or uracil. Various degrees of stringency of hybridization can be employed. The more severe the conditions, the greater the complementarity that is required for duplex formation. Severity of conditions can be controlled by temperature, probe concentration, probe length, ionic strength, time, and the like.

Preferably, hybridization is conducted under high stringency conditions by techniques well known in the art, as described, for example, in Keller, G. H. & M. M. Manak, DNA Probes, and the companion volume DNA Probes: Background, Applications, Procedures (various editions, including 2^{nd} Edition, Nature Publishing Group, 1993). Hybridization is also described extensively in the Molecular Cloning manuals published by Cold Spring Harbor Laboratory Press, including Sambrook & Russell, Molecular Cloning: A Laboratory Manual (2001). A non-limiting example of high stringency conditions for hybridization is at least about 6×SSC and 1% SDS at 65° C., with a first wash for 10 minutes at about 42° C. with about 20% (v/v) formamide in 0.1×SSC, and with a subsequent wash with 0.2×SSC and 0.1% SDS at 65° C. A non-limiting example of hybridization conditions are conditions selected to be about 1, 2, 3, 4, 5, 6, 7, 8, 9, 10, 11, 12, 13, 14, 15, 16, 17, 18, 19, 20, 21, 22, 23, 24, or 25° C. lower than the thermal melting point (Tₘ) for the specific sequence in the particular solution. Tₘ is the temperature (dependent upon ionic strength and pH) at which 50% of the target sequence hybridizes to a perfectly matched probe. Tₘ typically increases with [Na⁺] concentration because the sodium cations electrostatically shield the anionic phosphate groups of the nucleotides and minimize their repulsion. The washes employed may be for about 5, 10, 15, 20, 25, 30, or more minutes each, and may be of increasing stringency if desired.

Calculations for estimating Tₘ are well-known in the art. For example, the melting temperature may be described by the following formula (Beltz, G. A., K. A. Jacobs, T. H. Eickbush, P. T. Cherbas, and F. C. Kafatos, Methods of Enzymology, R. Wu, L. Grossman and K. Moldave [eds.] Academic Press, New York 100:266-285, 1983).

Tm=81.5° C.+16.6 Log [Na⁺]+0.41(%G+C)−0.61(%formamide)−600/length of duplex in base pairs.

A more accurate estimation of Tₘ may be obtained using nearest-neighbor models. Breslauer, et al., Proc. Natl. Acad. Sci. USA, 83:3746-3750 (1986); SantaLucia, Proc. Natl. Acad. Sci. USA, 95: 1460-1465 (1998); Allawi & SantaLucia, Biochemistry 36:10581-94 (1997); Sugimoto et al., Nucleic Acids Res., 24:4501-4505 (1996). Tₘ may also be routinely measured by differential scanning calorimetry (Duguid et al., Biophys J, 71:3350-60, 1996) in a chosen solution, or by other methods known in the art, such as UV-monitored melting. As the stringency of the hydridization conditions is increased, higher degrees of homology are obtained.

The term “subtelomere” or any grammatical variation thereof (e.g., subtelomeric region or subtelomeric DNA etc.), as used herein, refers to a chromosome region located immediately adjacent to the centromere side of the telomere repeat sequence (e.g., (TTAGGG)ₙ) at the chromosome terminus. Subtelomere generally contains telomeric repeat sequence interspersed with imperfect telomeric repeat and/or variable sequences.

Host cells can be infected with any of HHV-6 viruses, including HHV-6A and HHV-6B. In an embodiment, host cells are infected with HHV-6A strain U1102 (Accession No. X83413). In another embodiment, host cells are infected with HHV-6B Z29 strain.

Advantageously, the subject methods permit determination of a previously unknown subtelomere DNA sequence. In addition, as human subtelomeres are strikingly polymorphic in content, the subject invention would enable determination and analysis of allelic variations among normal and diseased individuals. As is exemplified in FIG. 11A-B, host chromosomal DNA of interest is first cleaved using restriction endonuclease molecules, thereby generating DNA fragments comprising HHV-6-subtelomere junctions. The resulting DNA fragments are self-circularized via intramolecular ligation to generate monomeric DNA circles, which serve as templates for IPCR amplification of HHV-6-subtelomere DNA.

Selection of appropriate restriction enzymes, which produce linear chromosomal DNA fragments prior to intramolecular circularization, can be determined empirically by Southern blotting and hybridization procedures. Southern Blot probes can be derived from part or all of the HHV-6 DR_{R} DNA sequences, telomere sequences, or known subtelomere sequences.

Suitable restriction endonuclease molecules include, for example, MboI, Mme I, HaeIII, FspBI, Csp6I, NciI, NsiI, PovII, Esp3I, PstII, ClaI, BssHII, TaqI, RsaI, Sau3AI, HindIII, EcoRI, EcoRII, BamHI, NotI, SamI, AluI, KpnI, PstI, ScaI, and XbaI. In an embodiment, MboI, a frequent cutter that cleaves methylated DNA, is employed. Preferably, the restriction endonuclease molecules used to produce the linear chromosomal DNA fragments prior to intramolecular circularization do not cleave part or all of HHV-6 right end direct repeat (DR_{R}) DNA.

Optimally, the DNA fragments comprising HHV-6-subtelomere junction, generated by restriction digestion of chromosomal DNA, are diluted and ligated under conditions that favor self-circularization of DNA fragments. Additionally, linear or circularized DNA comprising a HHV-6-subtelomere junction may be further selected, for example, by Southern hybridization using HHV-6, telomere or subtelomere probes.

IPCR primers can be derived from any of U1-U100 of HHV-6 right end direct repeat (DR_{R}) DNA sequence to initiate extension in the opposite direction of DR_{R}. In certain embodiments, the IPCR primers are derived from DR_{R} DNA of U94 (such as U94 rep gene), U53 or U54. In an embodiment, the IPCR primer of the subject invention is, or is complementary to, at least 10 contiguous nucleotides of one strand of HHV-6 DR_{R} sequence. In certain embodiments, the IPCR primer of the subject invention is, or is complementary to, at least 8, 9, 10, 11, 12, 13, 14, 15, 16, 17, 18, 19, 20, 21, 22, 23, 24, or 25 contiguous nucleotides of one strand of HHV-6 DR_{R} sequence. In a specific embodiment, the subject IPCR primer comprises SEQ ID NO:1 (IPCR-1) or SEQ ID NO:2 (IPCR-2). In a further specific embodiment, the subject IPCR primer is SEQ ID NO:1 (IPCR-1) or SEQ ID NO:2 (IPCR-2).

The IPCR products comprising HHV-6-subtelomere DNA can be further selected, isolated, and characterized using a combination of techniques in molecular biology, including gel electrophoresis, Southern blot, sequencing, restriction digestion, and cloning. For instance, IPCR products may be gel electrophoresed for visualization and purification of HHV-6-subtelomere DNA. In addition, IPCR products may be analyzed by Southern blotting using HHV-6 or telomere probes for identifying the presence of specific DNA sequences and/or site of HHV-6 integration. Further, restriction enzyme digestion may be employed to fragment IPRC products at specific sites, thereby constructing a restriction map of the telomere-subtelomere assembly.

Additionally, the precise sequence of the HHV-6-subtelomere DNA may be determined using DNA sequencing techniques, such as for example, dideoxy sequencing reactions (Sanger sequencing), sequencing by synthesis using reversibly terminated labeled nucleotides, primer walking, shotgun sequencing, gel electrophoresis sequencing, multi-color fluorescence-based DNA techniques, sequencing by hybridization, DNA microarray, pyrosequencing, 454 sequencing (Roche) (Margulies, M et al. 2005, Nature, 437, 376-380), polony sequencing, and SOLiD sequencing.

### Kits

Another aspect of the subject invention provides kits for rapid and accurate determination of subtelomere DNA sequence as well as the telomeric site of HHV-6 integration. In an embodiment, the kit comprises an IPCR primer of the subject invention, useful for amplification of the subtelomere DNA. Specifically embodied herein are IPCR primers comprising SEQ ID NO:1 (IPCR-1) or SEQ ID NO:2 (IPCR-2).

Optionally, the kit may include any material useful for performing any step of the subject invention as described above. For instance, the kit may further comprise any material useful for determination and/or analysis of the telomere and/or subtelomere DNA of interest. For instance, the kit may comprise primers for chromosomal-specific PCR, chromosomal-specific hybridization probes, hybridization probes for identifying HHV-6, telomere and/or subtelomere sequences, restriction enzyme molecules, DNA ligase (e.g., T4 DNA ligase), oligonucleotides derived from host mitochondrion DNA, Taq DNA polymerase, and primers for amplification of viral, telomere and/or subtelomere DNA. In an embodiment, the kit comprises HHV-6-, telomere- and/or subtelomere-specific probes for identification of HHV-6-subtelomere DNA. In a specific embodiment, the kit may further comprise any oligonucleotide molecules comprising any of SEQ ID NO:3-SEQ ID NO:40.

The kit may also comprise, e.g., a buffering agent, a preservative, or a stabilizing agent. Each component of the kit is usually enclosed within an individual container and all of the various containers are within a single package along with instructions.

### Applications

In a further aspect, based on HHV-6-subtelomere DNA sequences determined using the methods of the subject invention, high-resolution sequence maps of human subtelomere regions, much of which remain uncharacterized, can be constructed. In addition, the precise location into which HHV-6 DNA is inserted in telomeres of HHV-6-infected individuals can be determined. The subject methods can also be used to generate a genomic DNA library of subtelomeric region. Further, cDNA library of coding regions of subtelomere can be generated from subtelomeric transcripts. Thus, genomic and cDNA libraries of human subtelomeric regions stored on 23 genetically distinct chromosomal pairs can be derived based on the subject IPCR-based methods.

In addition, the determination of the subtelomere sequences provides an essential starting point for identification and analysis of subtelomere genes. These genes can serve as biomarkers for diagnosis and prognosis of diseases, or alternatively, as targets for development of novel therapeutics. The subject invention also allows for determination of allelic variations of subtelomere sequences among individuals.

In an embodiment, the subject invention can be used to determine a subtelomere sequence of an individual for diagnosis of diseases associated with subtelomere DNA mutation (e.g., substitution, addition, deletion, inversion and translocation), which would provide insights to pathogenesis and progression of a range of subtelomere-related diseases, such as for example, 9q subtelomeric deletion syndrome and subtelomeric chromosome rearrangements associated with idiopathic mental retardation.

In addition, the subject invention allows for determination of the telomeric site into which HHV-6 genome DNA integrates in HHV-6 infected individuals. Chromosomal integration of HHV-6A has been reported as causally associated with progression of AIDS, graft rejection, neurological diseases such as multiple sclerosis (MS), chronic fatigue syndrome (CFS), brain tumor, congenital infection, connective tissue diseases, pediatric cardiomyopathy, epilepsy, anemia, drug-induced hypersensitivity syndrome, and cancer. Determining the precise telomeric site of HHV-6 integration would facilitate the discovery of, for example, the viral and host molecular mechanism during telomere-specific HHV-6 integration, viral and cellular components critical for telomere-specific HHV-6 integration, and their implication on pathogenesis of diseases.

Specifically, HHV-6A is suggested as a co-factor in the progression of AIDS and other immunosuppression-related diseases (Lusso et al., 1989, 1991, 2007, Takahashi et al., 1989, Griffiths et al., 2000, Kidd et al., 2000). HHV-6A reactivates during immunosuppression, leading to speculation that the virus suppresses T cell function and cooperates with HIV. The mechanism by which HHV-6A induces immunosuppression is unknown. Identification of the telomeric site of integration in individuals will provide insights into how HHV-6 genome interferes with transcription or translation of telomere/subtelomere genes in these sites.

Telomeric sites of HHV-6 integration determined using the subject method can be further analyzed to elucidate the interaction of subtelomere genes with HHV-6 genome inserted in specific telomeric sites. Examples of genetic research schemes on telomere and subtelomere are described in Examples 7-17. For instance, using the subject method, the present inventors discovered that the right end of the HHV-6 genome is near the subtelomere of the chromosome (Arbuckle et al., 2010); suggesting that the viral genome may interfere with telomeric-repeat-containing RNA (TERRA) transcription.

In an embodiment, the subject invention will also lead to the identification and analysis of viral genes associated with the induction and maintenance telomere-specific integration. In another embodiment, the subject invention allows identification of telomeric other chromosomal sites recognized by HHV-6 viruses during integration. In another embodiment, the subject invention can be used to identify chromosomes and telomeric sites into which HHV-6 is frequently inserted.

### Materials and Methods

### Patients.

Peripheral blood samples of four independent families were obtained from the HHV-6 Foundation, after the subjects had given informed consent. Of each family, more than one family member, including a parent and at least one child, had at least one million copies of HHV-6 per ml of peripheral blood (Table 1). Several individuals were suffering from neurological symptoms, whereas others were asymptomatic.

TABLE 1
Patients from Four Independent Families with Chromosome Integrated HHV-6

| | | | | HHV-6 O- | | | Percent Sequence Identity to | |
|---|---|---|---|---|---|---|---|---|
| Family | | | | PCR^{a} | HHV-6 | Chromosome | HHV-6A (U1102) | |
| Number | Age, Sex | Subject | Disease Stage | (copies/ml) | Subtype^{b} | HHV-6 FISH | U94 | DR |
| 1 | 58, M | Father | Asymptomatic | 629,000 | A | 18q23 | 98% | 98% |
| 1 | 54, F | Mother | PCR Negative | Negative | Negative | n/a | n/a | n/a |
| 1 | 24, M | Sibling-1 | Asymptomatic | 1,400,000 | A | 18q23 | 98% | Not done |
| 1 | 22, F | Sibling-2 | CNS Dysfunction, hypersomnia | 1,700,000 | A | 18q23 | 98% | Not done |
| 1 | 12, M | Sibling-3 | CNS Dysfunction, ataxia | 1,600,000 | A | 18q23 | Not done | Not done |
| 2 | 80, F | Mother | Mild dementia | 625,000 | A | 17p13.3 | 98% | Not done |
| 2 | 45, F | Sibling-1 | CNS Dysfunction, fatigue | 4,100,000 | A | 17p13.3 | 98% | 98% |
| 3 | 76, M | Father | Asymptomatic | 2,000,000 | B | 22q | Not done | Not done |
| 3 | 61, F | Mother | PCR Negative | Negative | Negative | n/a | n/a | n/a |
| 3 | 34, M | Sibling-1 | CNS Dysfunction, fatigue | 2,000,000 | B | 22q | Not done | Not done |
| 4 | 62, F | Mother | Asymptomatic | 4,000,000 | B | Not Done | Not done | Not done |
| 4 | 36, M | Sibling-1 | Asymptomatic | 4,500,000 | B | Not Done | Not done | Not done |
| 4 | 29, F | Sibling-2 | CNS Dysfunction, fatigue, ataxia | 4,200,000 | B | Not Done | Not done | Not done |

^{a}O-PCR on whole blood completed by ViseCor Laboratories, Lee’s Summit MO
^{b}Subtypes were determined using PCR with subtype-specific primers

### Primary T Cells, Cell Lines, and Viruses.

PBMCs were isolated from peripheral blood samples using Lymphoprep™ according to the manufacturer's protocol. PBMCs were then incubated in RPMI-1640 medium containing 10% FBS and 5 μg/ml PHA (Sigma-Aldrich) for 72 hours followed by culturing in 100 U/μl IL-2 medium.

T-cell lines Jjhan (HHV-6 Foundation), Molt3 (ATCC), and Burkitt's lymphoma cell line Raji (ATCC) were maintained in RPMI-1640 medium containing 10% FBS. Human embryonic kidney-293 cells (HEK-293) were maintained in Dulbecco's modified Eagle's medium (DMEM) supplemented with 10% FBS.

HHV-6A (U1102 strain) and HHV-6B (Z29 strain) viruses were obtained from P. Pellett (Wayne State University). HHV-6 integrated Burkitt's lymphoma cell line Katata was obtained from M. Daibata (Kochi Medical School, Japan) (37). Immortalization of patients' T-cells with Herpesvirus saimiri strain 484-77 was performed using techniques known in the art, as described in (38, 39).

### Oligonucleotides for PCR and ³²P-Labeled Probes

Primers were designed according to the GenBank consensus sequence for HHV-6A (U1102 Accs #X83413).

Oligonucleotides for inverse PCR (IPCR) and ³²P-labeled probes are:

IPCR-1:
(SEQ ID NO: 1)
5′GCACAACCCACCCATGTGGTAGTCGCGG3′;
IPCR-2:
(SEQ ID NO: 2)
5′CGTGTGTACGCGTCCGTGGTAGAAACGCG3′;
IPCR-probe:
(SEQ ID NO: 3)
5′CTTACACTTGCCATGCTAGC3′;
and
Telomere repeat probe:
(SEQ ID NO: 4)
5′TTAGGGTTAGGGTTAGGGTTAGGG3′.

Oligonucleotides for chromosomal-specific PCR and ³²P-labeled probes are:

HHV-6 DR_{R}:
(SEQ ID NO: 5)
5′CATAGATCGGGACTGCTTGAAAGCGC3′;
HHV-6 DR_{L}:
(SEQ ID NO: 6)
5′CTTTCTCGCTGTGCCTCACGCTGTC3′;
Chromosome 11q primer:
(SEQ ID NO: 7)
5′CAGACCTTGGAGGCACGGCCTTCG3′ (33);
Chromosome 17p primer:
(SEQ ID NO: 8)
5′AACATCGAATCCACGGATTGCTTTGTGTAC3′ (33);
Chromosome 18q primer:
(SEQ ID NO: 9)
5′CTCATGTCCTCGGTCTCTTGCCTC3′;
HHV-6 DR_{R}-probe:
(SEQ ID NO: 10)
5′GCGGAGACACATAGCCTTGGCGGGAAGAC3′;
and
Chromosome 17p probe:
(SEQ ID NO: 11)
5′CCCAAGCAGGTTGAGAGGCTGAGG3′.

Oligonucleotides for direct repeat (DR) (U1102 421-1474, 151654-152707) are:

DR-1:
(SEQ ID NO: 12)
5′TGCCGCTTCAACTTCACCTT3′;
and
DR-2:
(SEQ ID NO: 13)
5′AGATGTGGAGAGAAACGCGA3′.

Oligonucleotides for ORF U94 (U1102 141267-143197) are:

U94L-1:
(SEQ ID NO: 14)
5′TGTTCTTCTGCTAACTCGGACGCA3′;
U94L-2:
(SEQ ID NO: 15)
5′CAGTTCCAATGGGCGTGGACAAAT3′;
U94R-1:
(SEQ ID NO: 16)
5′ATCCACGCGTCTTCCGTGACTATT3′;
and
U94R-2:
(SEQ ID NO: 17)
5′TGTTCATGTCTTCCGGCGAAAGGT3′.

Mitochondrion oligonucleotides (41) are:

2R:
(SEQ ID NO: 18)
5′TGGACAACCAGCTATCACCA3′;
7F:
(SEQ ID NO: 19)
5′ACTAATTAATCCCCTGGCCC3′;
7R:
(SEQ ID NO: 20)
5′CCTGGGGTGGGTTTTGTATG3′;
13F:
(SEQ ID NO: 21)
5′TTTCCCCCTCTATTGATCCC3′;
13R:
(SEQ ID NO: 22)
5′GTGGCCTTGGTATGTGCTTT3′;
17F:
(SEQ ID NO: 23)
5′TCACTCTCACTGCCCAAGAA3′;
17R:
(SEQ ID NO: 24)
5′GGAGAATGGGGGATAGGTGT3′;
22F:
(SEQ ID NO: 25)
5′TGAAACTTCGGCTCACTCCT3′;
and
22R:
(SEQ ID NO: 26)
5′AGCTTTGGGTGCTAATGGTG3′.

HHV-6A GFP primers are:

U53L:
(SEQ ID NO: 27)
5′GGCCCGCGGAGTAGTTCCGCGTCAGAATGC3′;
U53R:
(SEQ ID NO: 28)
5′GGCGGATCCCATTCGTTTTATTGAACGCG3′;
U54L:
(SEQ ID NO: 29)
5′GGCAAGCTTGCAATGGTTAAAAGTTGTTTTTTG3′;
and
U54R:
(SEQ ID NO: 30)
5′GCGCCGCGGCTGCATACTTGCTACCGGAAC3′.

pEGFP-N2 vector primers are:

GFP-N2-L:
(SEQ ID NO: 31)
5′CGCGGATCCATTAATAGTAATCAATTACGG3′;
and
GFP-N2-R:
(SEQ ID NO: 32)
5′CGCGGATCCCGCCTTAAGATACATTGATGAG3′.

Oligonucleotides for CsCl/Ethidium bromide gradient are:

### C484 Stp-F: 5′CTCAGAACGCGGCAACAAACTTGA3′ (SEQ ID NO:33);

### C484 Stp-R: 5′TTTCGGCATACCTGGATCCCATGA3′ (SEQ ID NO:34);

Cytochrome-C oxidase-F: 5′TTCGCCGACCGTTGACTATT3′(SEQ ID NO:35);
Cytochrome-C oxidase-R: 5′AAGATTATTACAAATGCATGGGC3′ (SEQ ID NO:36) (Cote et al., Changes in mitochondrial DNA as a marker of nucleoside toxicity in HIV-infected patients. N Engl J. Med. 2002.);
Beta actin-F: 5′CTGGAACGGTGAAGGTGACA3′ (SEQ ID NO:37); and
Beta actin-R: 5′AAGGGACTTCCTGTAACAATGCA3′ (SEQ ID NO:38) (Vandesompele et al., Accurate normalization of real-time quantitative RTPCR data by geometric averaging of multiple internal control genes. Genome Biology. 2002.).

Primers for U94 qPCR are:

U94 qPCR-1:
(SEQ ID NO: 39)
5′ACAGCCATTCGATGGTTCCCAGAA3′;
and
U94 qPCR-2:
(SEQ ID NO: 40)
5′AACGAACTGGGAGACGTATGCGAT3′.

U94 qPCR conditions

TABLE 2
U94 qPCR Cycling Conditions

| | | |
|---|---|---|
| Cycles | Temperature (° C.) | Time |

| | | | |
|---|---|---|---|
| 1 | 94 | 2 | min |
| 40 | 94 | 15 | sec |
| | 60 | 1 | min |
| | 72 | 30 | sec |

### Sequencing ORF U94 and Direct Repeat (DR)

HHV-6 sequences present in PMBCs of family members were amplified by primers spanning ORF U94 (fragment 141,267-143,197, primers U94L-1 and U94L-2; U94R-1 and U94R-2) using 300 ng of genomic DNA with REDTaq DNA polymerase (Sigma-Aldrich). Cycling conditions were 95° C. for 1 min, 66° C. for 1 min, and then 72° C. for 1.5 min for a total of 24 cycles. Amplification of DR (fragment 421-1474) was performed with primers DR-1 and DR-2 and under cycling conditions of 95° C. for 1 min, 53° C. for 1 min, and 72° C. 1.5 min. DNA bands were isolated from 0.8% agarose gel and cloned into pCR®4-Topo vector using the TOPO TA Cloning® Kit for Sequencing (Invitrogen). Clones (n=4 each region) were then sequenced using ABI Prism® 3100 Genetic Analyzer (Applied Biosystems).

### Inverse PCR (IPCR)

The procedure for amplification of the integration site of HHV-6 by IPCR, adapted from Ochman et al., is illustrated as follows (30). Briefly, 2 μg of patient genomic DNA was restriction digested with 10 U of MboI (Promega) for 14 hrs at 37° C. To allow monomeric circularization and prevent ligation of several fragments, MboI-digested DNA was diluted to 2 μg/ml and incubated with 0.045 U/μl T4 DNA ligase (Promega) for 14 hrs at 15° C. Amplification of 100 ng of genomic DNA was performed with Expand 20 kbPLUS PCR System (Roche) and primers IPCR-1 and IPCR-2.

### Reactivation of HHV-6A.

Freshly isolated PBMCs at 1×10⁶ cells per ml cell concentration were cultured in RPMI-1640 medium supplemented with 10% FCS, 20 ng/ml TPA, and 1×10⁻⁶ M hydrocortisone or 80 ng/ml TSA for 3 to 5 days. To isolate reactivated virus, 1×10⁴ Molt-3 cells were added to 10⁶ of PBMC in 1 ml and cultured for 10-14 days. Reactivation was monitored for cell CPE, Gardella gel, qPCR, and sequencing of ORF U94.

### Chromosome-Specific PCR.

Chromosome-specific PCR for determining integration of HHV-6 in chromosome 17p was performed using 400 ng genomic DNA and primers DR_{R} and 17p or DR_{L} and 17p (28). Co-hybridization of PCR products with HHV-6 and telomere sequences was performed using Southern hybridization.

### CsCl/Ethidium Bromide Gradient.

DNA (50 μg) isolated from HEK-293 and T-cells of family members with chromosomally integrated HHV-6 were subjected to centrifugation at 45,000 rpm for 72 hrs in a solution of CsCl/ethidium bromide (density=1.55 g/ml). Covalently closed circular DNA and linear DNA fractions were visualized by agarose gel electrophoresis then subjected to amplification with primers to HHV-6 ORF-U94, beta actin, and cytochrome C oxidase.

### Southern Hybridization.

Vacuum blotting and hybridization with ³²P-labeled HHV-6A (U1102) cosmids PMF311-12 and PMF335-6 (40) were performed using techniques known in the art, as described in (44).

Following are examples that illustrate embodiments for practicing the invention. These examples should not be construed as limiting. All percentages are by weight and all solvent mixture proportions are by volume unless otherwise noted.

### Example 1

### Determination of Telomeric Site of HHV-6 Integration

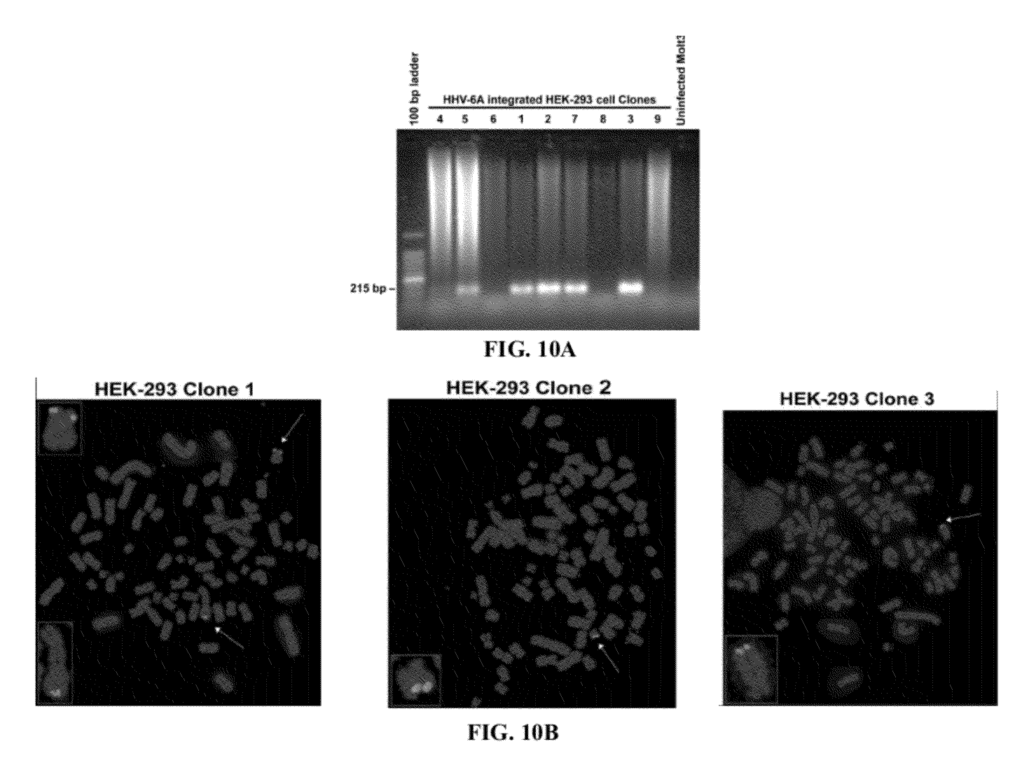

This Example illustrates methods for determining telomeric sites in which HHV-6 genomic DNA is inserted. Briefly, HEK-293 cells at 50% confluency were infected with HHV-6A (U1102) at 0.1 multiplicity of infection (MOI). After incubation for five days, the infected cells were washed to remove extracellular virus. Single cells were introduced into a 96-well plate and expanded. The results of ORF U94-specific PCR showed that 10 out of 22 clones have viral genome (FIG. 10a). Three PCR-positive clones and one PCR-negative clone were examined by FISH to determine the integration of HHV-6 genomic DNA. In two PCR-positive clones, FISH analysis identified integrated HHV-6A in one chromosome. In the third PCR-positive clone, the virus had integrated into two chromosomes (FIG. 10b).

The specific chromosomes into which HHV-6 had integrated in the HEK-293 cells was not identified using standard cytogenetic methods, due to the aneuploidy and chromosomal rearrangements present in these cells. Therefore, novel methods for determining site of integration were developed using inverse PCR (IPCR) (30).

The procedure for determining telomeric site in which HHV-6 genomic DNA is inserted is illustrated in detail in FIGS. 11A-B. Briefly, chromosomal DNA of HHV-6-infected cells was isolated and restriction digested with the frequent cutter MboI, which cleaves methylated DNA. After digestion and heat inactivation, DNA was diluted to 2 μg/ml, and T4 ligase was added to enable self-circularization of the HHV-6 sequence and adjacent chromosomal fragment. The ligated DNA was amplified using primers designed from the extreme right end of the viral genome (FIG. 11a). Southern blotting identified IPCR products that hybridized with a human telomere probe as well as an HHV-6 probe from integrated HEK-293 and patient T-cells (FIG. 11b). No hybridization was observed without ligation of the MboI fragments. In summary, the IPCR procedure detects the integration of HHV-6A genome into the telomeres of host cells (e.g., in vitro infected HEK-293 cells). Further, DNA sequences of a telomeric site in which HHV-6 genomic sequence is integrated can be detected by sequencing IPCR products using sequencing techniques known in the art.

### Example 2

### Determination of Chromosomal Integration of HHV-6 DNA Using Fish

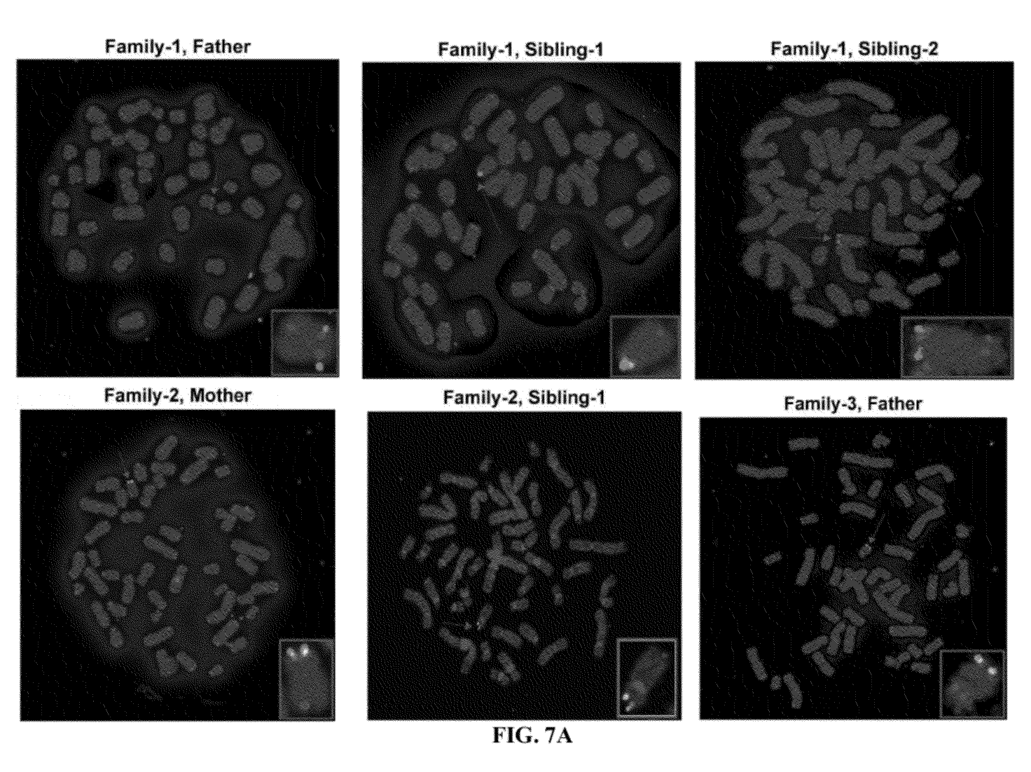

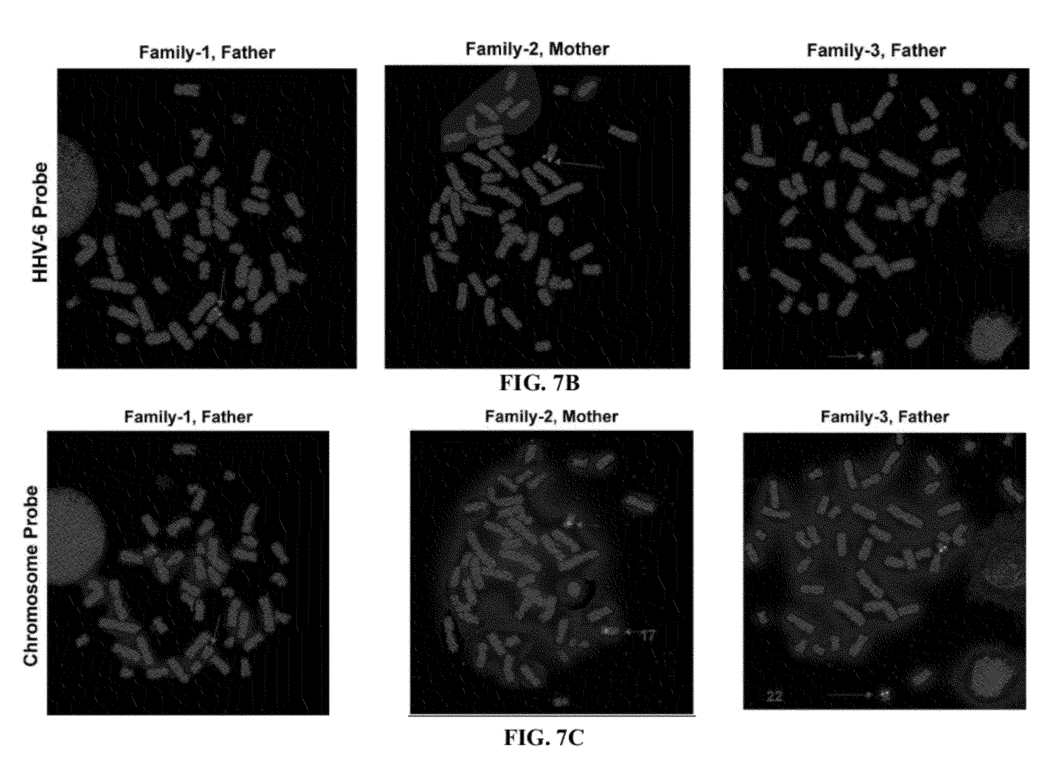

This Example illustrates methods for determining whether viral genomic DNA is integrated into the host chromosomes. Briefly, fluorescence in situ hybridization (FISH) was performed in T-cell cultures derived from peripheral blood samples. PBMCs were stimulated for 72 hours with 20 ng/ml PHA, and then cultured in RPMI 1640 medium containing 50 U/ml IL-2 and 10% FCS. Metaphase chromosomes were generated according to standard cytogenic protocol (Kowalska et al., Chromosome Research, 2007), stained with DAPI and hybridized with various probes as follows: FITC-conjugated HHV-6A (U1102 strain) cosmid probes pMF311-2, pMF335-6 (green) (40), and cy5-conjugated telomere peptide nucleic acid (PNA) probe (red) (DakoCytomation) (FIG. 7). The experiments were performed in two independent laboratories (University of Minnesota, Minneapolis, Minn. and Children's Cancer Research Institute, Vienna, Austria). Each laboratory was blind to knowledge of the individual and family membership from which each specimen had been obtained.

FIG. 7 shows HHV-6-specific fluorescence detection in association with the telomeric regions of chromosomes (Table 1 and FIG. 7). The viral genome was present in the same chromosome of the corresponding parents and siblings, suggesting germ line transmission (26): Family-1 chromosome 18q23; Family-2 chromosome 17p13.3; and Family-3 chromosome 22q. The specific chromosome of HHV-6 integration was identified by co-hybridization with viral and chromosome specific probes. Of three families, HHV-6-specific FISH signal was detected in chromosomes 17p13.3, 18q23 and 22q13.3, respectively. In addition, HHV-6 and telomere FISH signals overlapped (FIG. 7).

Chromosomal integration of HHV-6 DNA, instead of a mere association of the telomere with episomal viral DNA, can be further confirmed by analyzing T-cells using the method of Gardella et al. (27). The Gardella method uses a vertical agarose gel capable of distinguishing cellular genomic DNA from covalently closed circular DNA (episomes) and from replicating linear viral DNA.

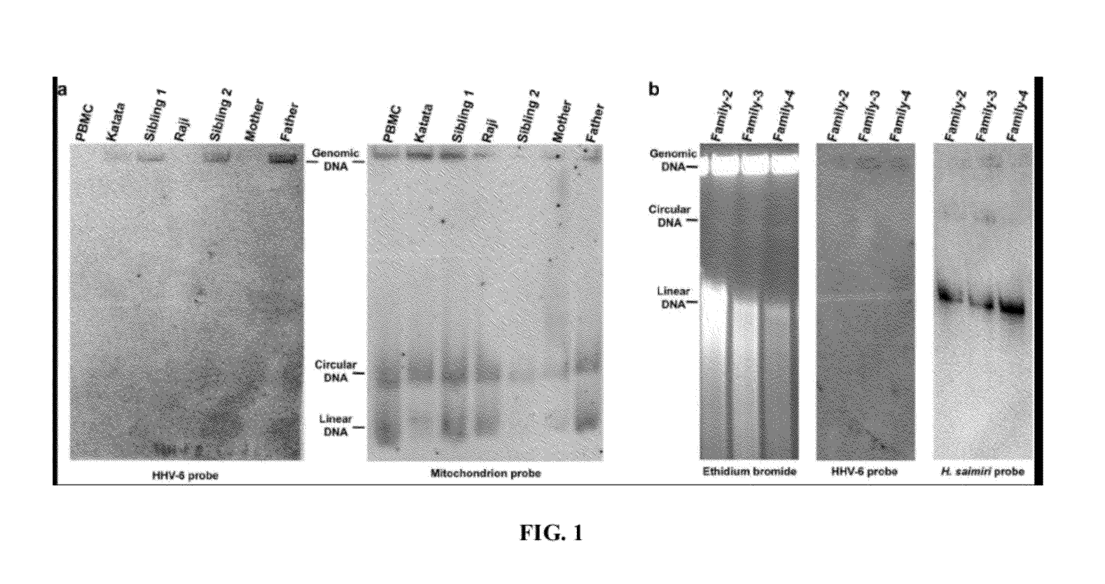

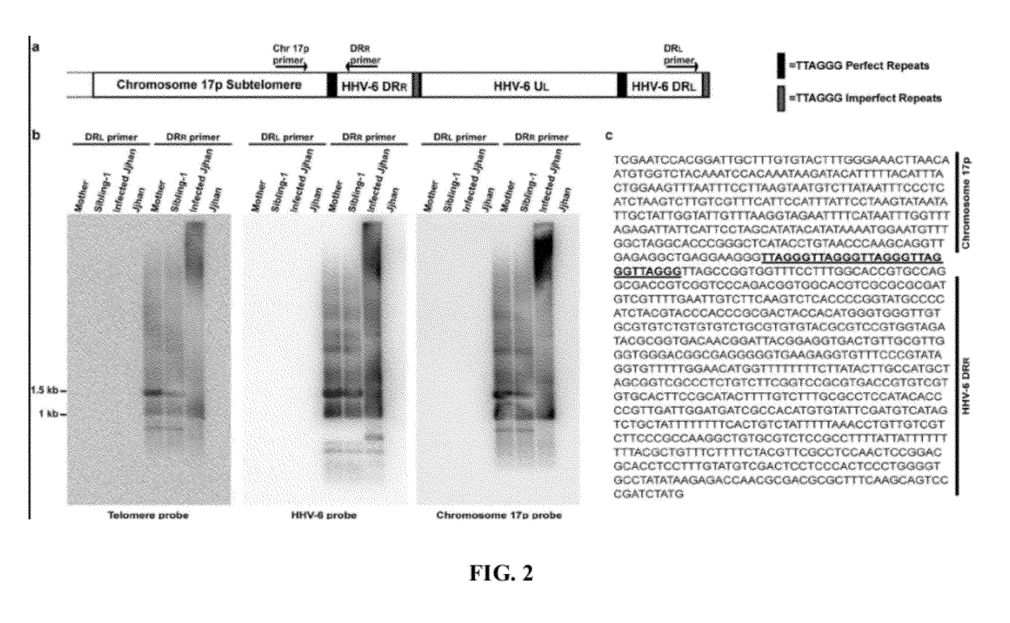

Specifically, one million T-cells were isolated from Family-1 members, control uninfected PBMCs, and HHV-6 positive Katata cell line (HHV-6B integrated Burkitt's lymphoma cell line) (37). Cells were loaded on a vertical agarose gel and analyzed for episomal, linear, or integrated DNA by the method of Gardella et al. (21). Southern hybridization with HHV-6 cosmid probe (FIG. 1a) confirmed the association of the viral genome with cellular DNA located in the loading well of these gels. No circular episomal and unit length linear viral DNA was detected in these experiments.

To further validate the above Gardella gel results, T-cells from three members of Family-1 were immortalized using Herpesvirus saimiri (HVS) strain C484. Southern hybridization with HHV-6 detected signals within the genomic fraction (loading well) of the Gardella gel. Hybridization of the same Southern blot with HVS probe detected both episomal circular and replicating linear HVS DNA, confirming proper release of herpes virus DNA in the gel (FIG. 1b).

### Example 3

### Confirmation of Chromosomal Integration of HHV-6 Genomic DNA by PCR

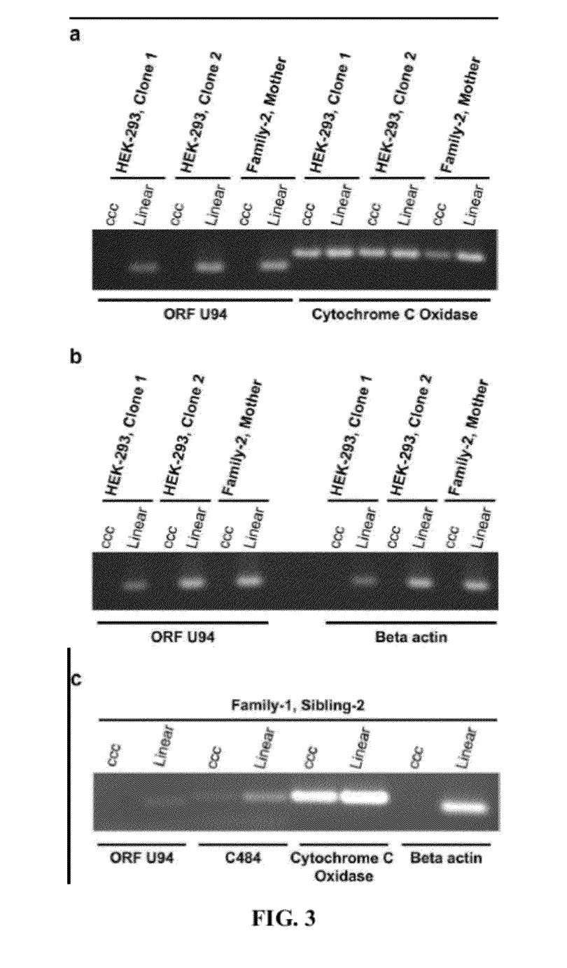

This Example confirms that HHV-6 genomic DNA integrates into chromosomes of host cells during viral infection. The majority of herpesviruses establish latency via circular nuclear episomes. However, as shown in Example 2, no HHV-6 episome is detected in host cells using the Gardella assay. To confirm that HHV-6 DNA only integrates into host chromosomes, a highly sensitive PCR assay was employed to detect the presence of small numbers of episomes, which might not have been detectable by the Gardella assay. Briefly, DNA was isolated from HHV-6A integrated HEK-293, T-cells from a member of Family-2, and T-cells from a member of Family-1 immortalized using HVS strain C484. The isolated DNA was subjected to CsCl/ethidium bromide gradient ultracentrifugation (FIG. 3). HHV-6 DNA was detected only in the linear fraction, and no episomal (ccc) DNA fraction was detected. Since the present PCR assay can detect as few as 1-5 molecules, it is confirmed that no viral episome was formed during HHV-6 infection. The detection of mitochondrial sequence in the linear fraction is expected since replicating and relaxed mitochondrial episomes band together with chromosomal linear DNA.

### Example 4

### Determination of Chromosomal Integration Site of HHV-6 DNA Using PCR

This Example illustrates methods for determination of chromosomal integration site of HHV-6 DNA. Briefly, the putative viral-chromosomal DNA junction is amplified using a primer pair homologous to the DR_{L} and DR_{R} of the viral genome and a primer to the subtelomere of chromosome 17p (FIG. 2a). Chromosomal DNA from Family-2 was analyzed since FISH had identified the integration of HHV-6 into chromosome 17p (FIG. 7) in this family, and the sequence of telomere-subtelomere junction of chromosome 17p is known (28). Amplification with the primer pair designed from DR_{R} and 17p subtelomere successfully amplified the viral-cellular junction, which is determined by co-Southern hybridization with HHV-6, telomere, and chromosome 17p oligonucleotide probes (FIG. 2b). In addition, the absence of amplification with a primer derived from DR_{L} confirmed the integration of HHV-6 into human subtelomere, which is mediated through the perfect TTAGGG found at the end of DR_{R}.

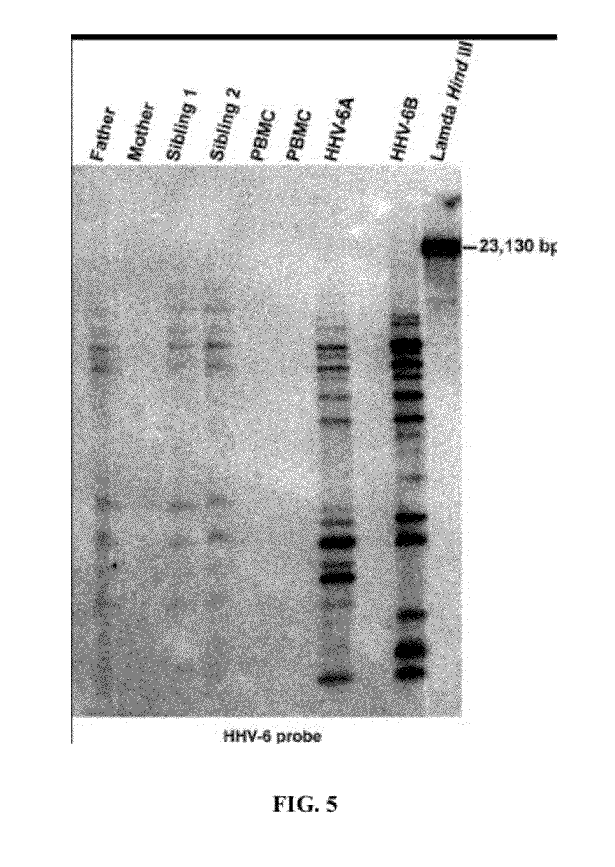

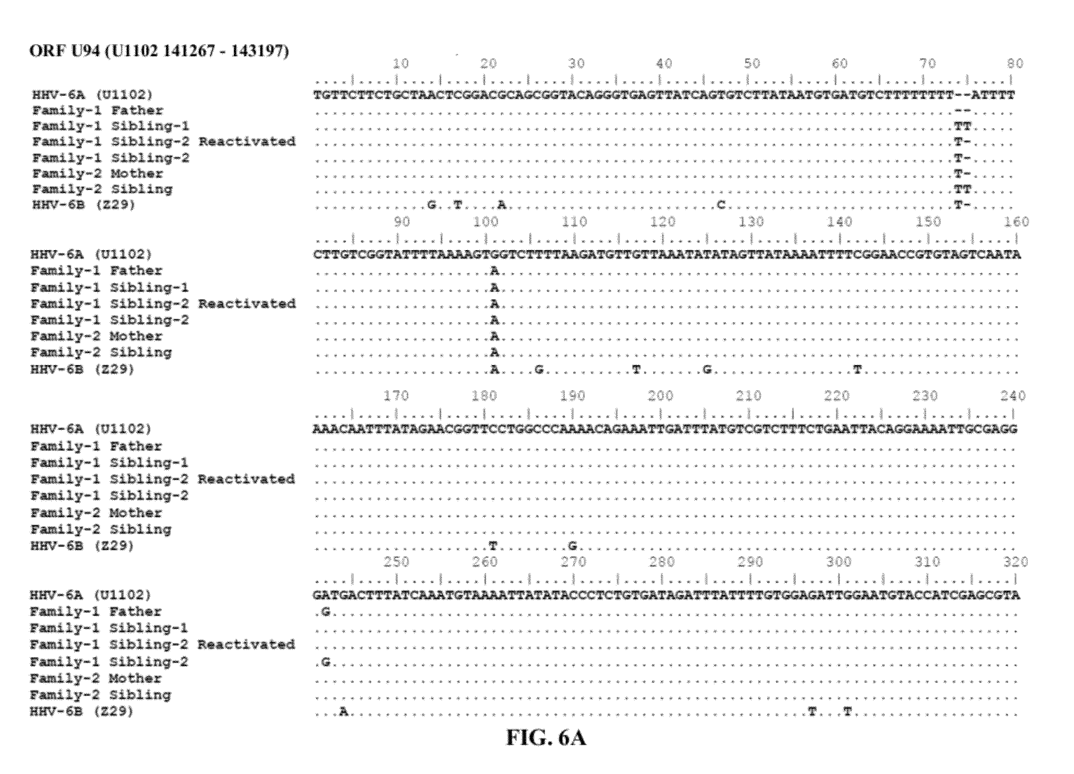

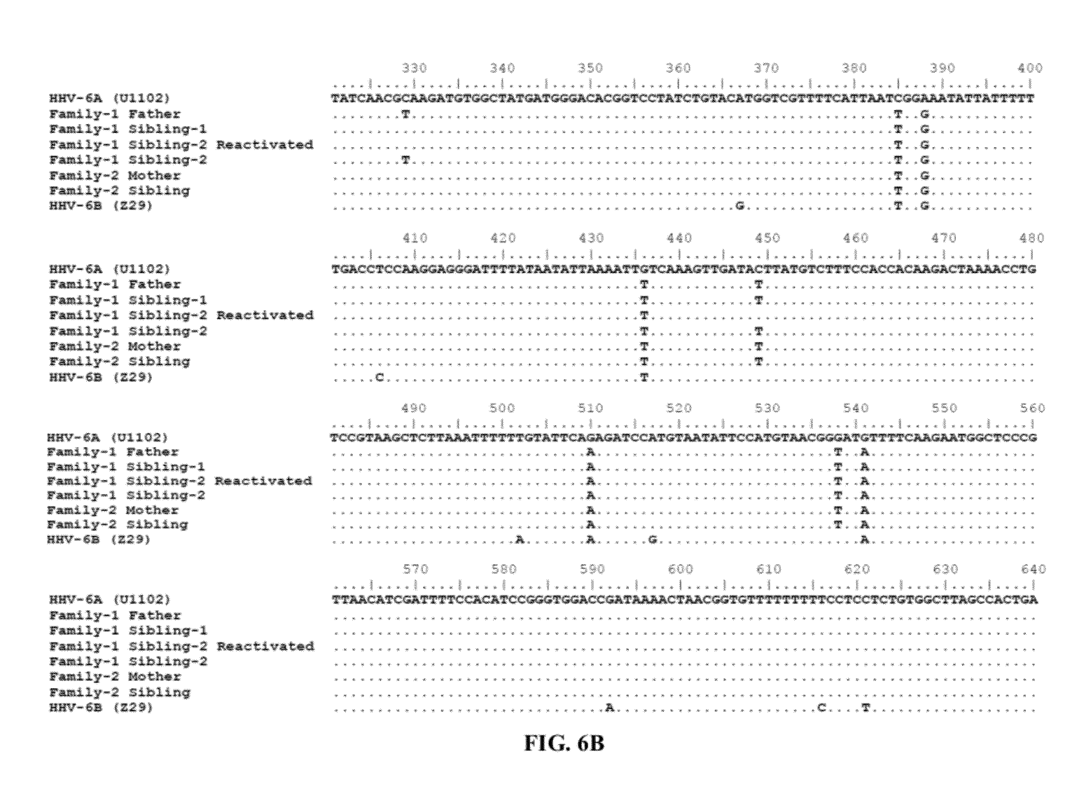

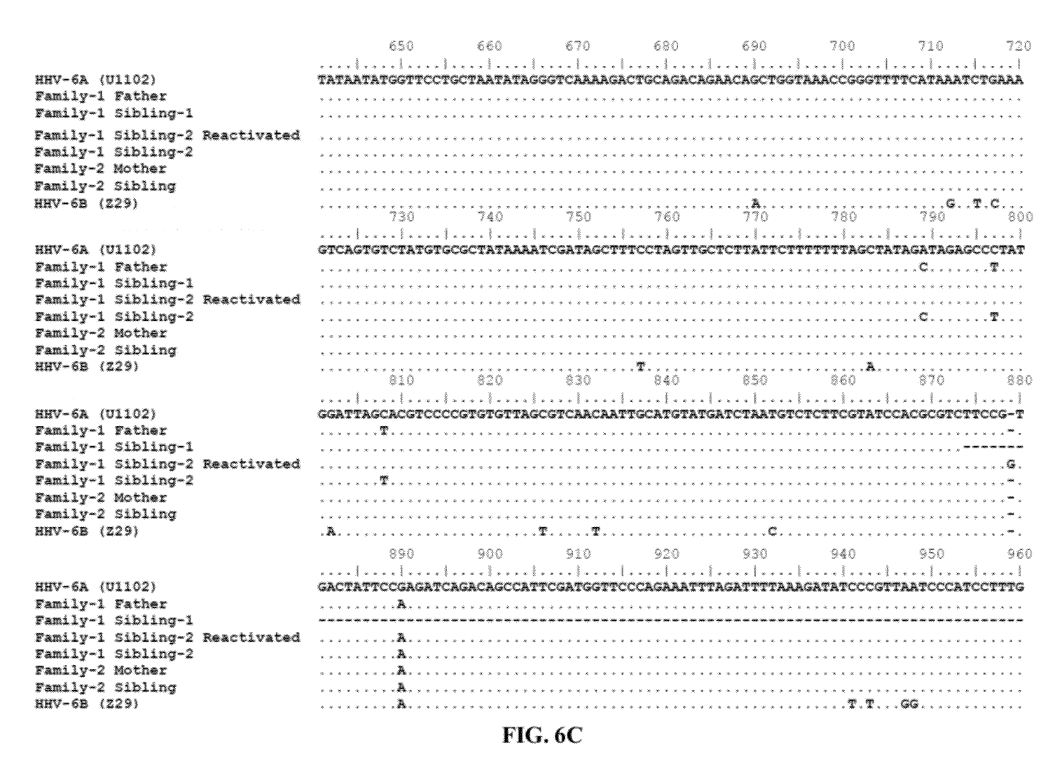

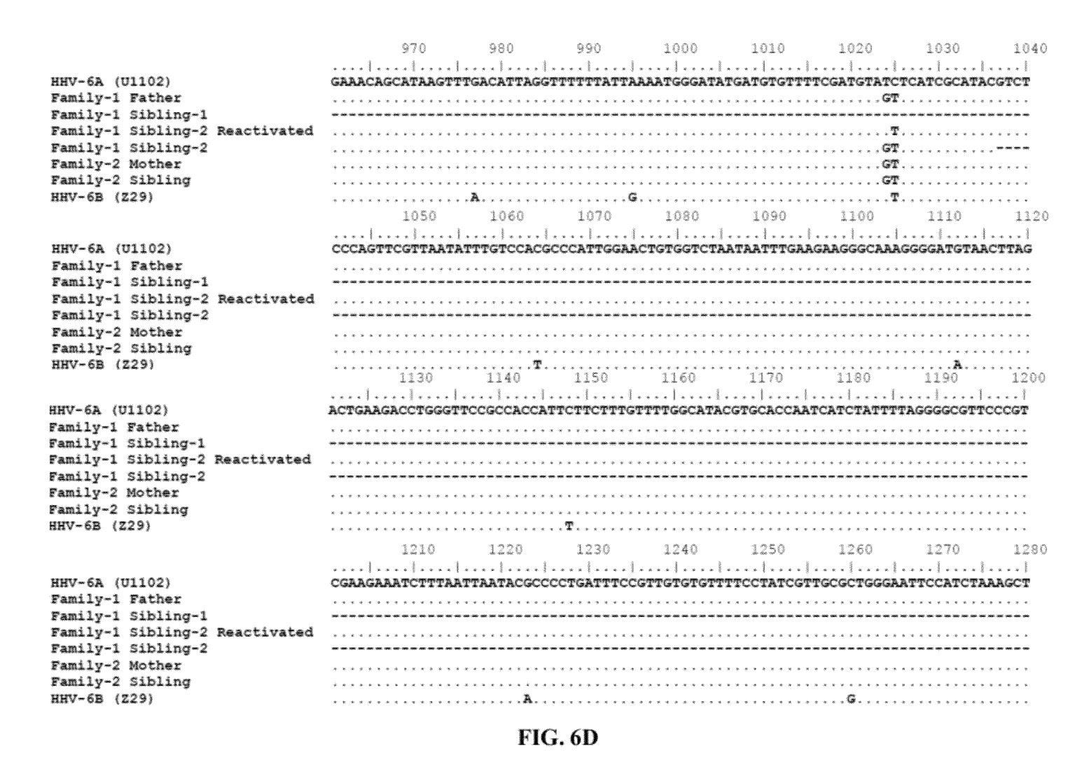

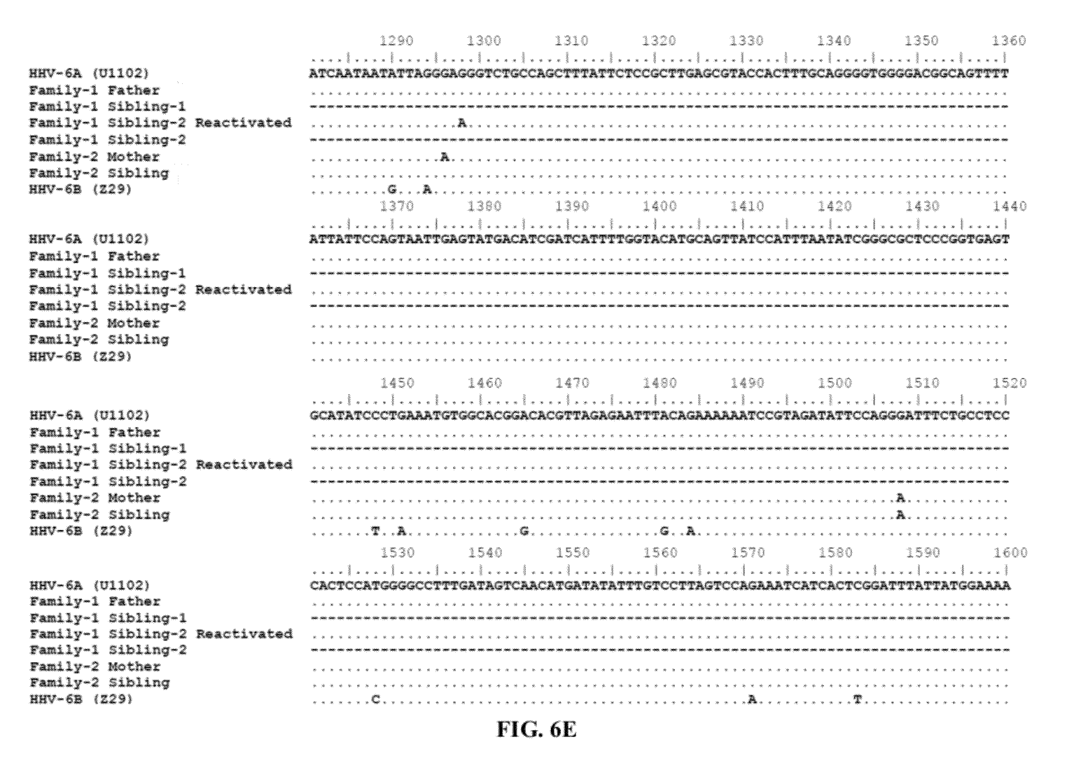

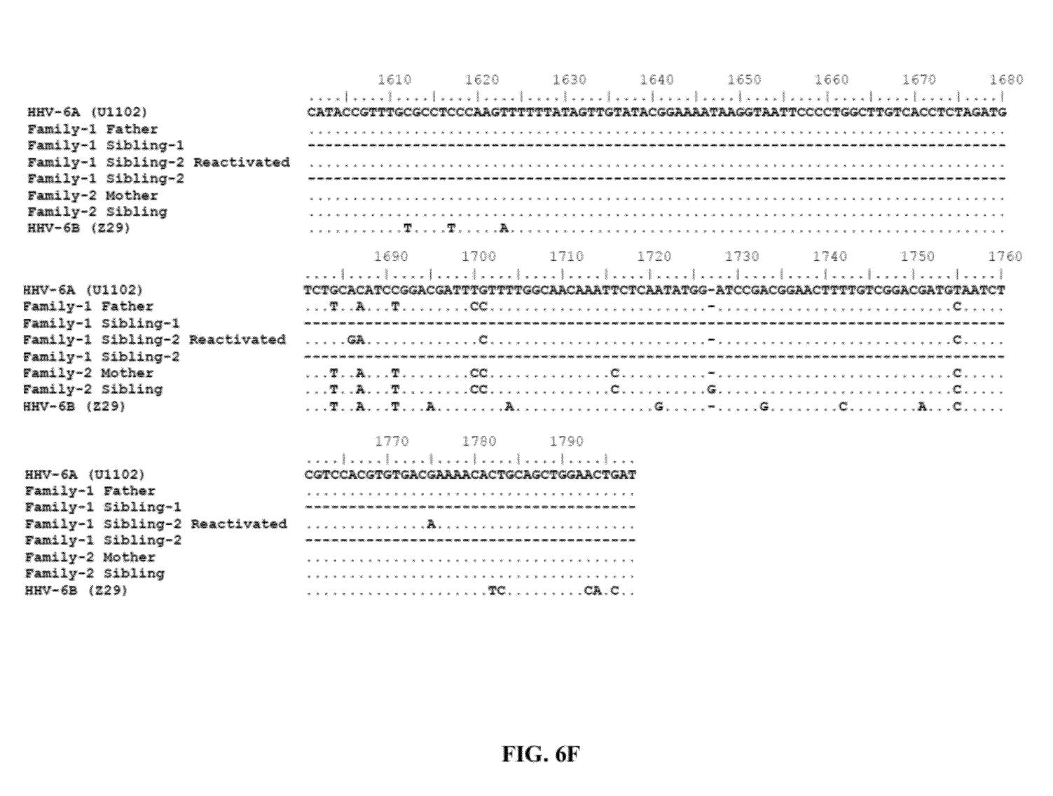

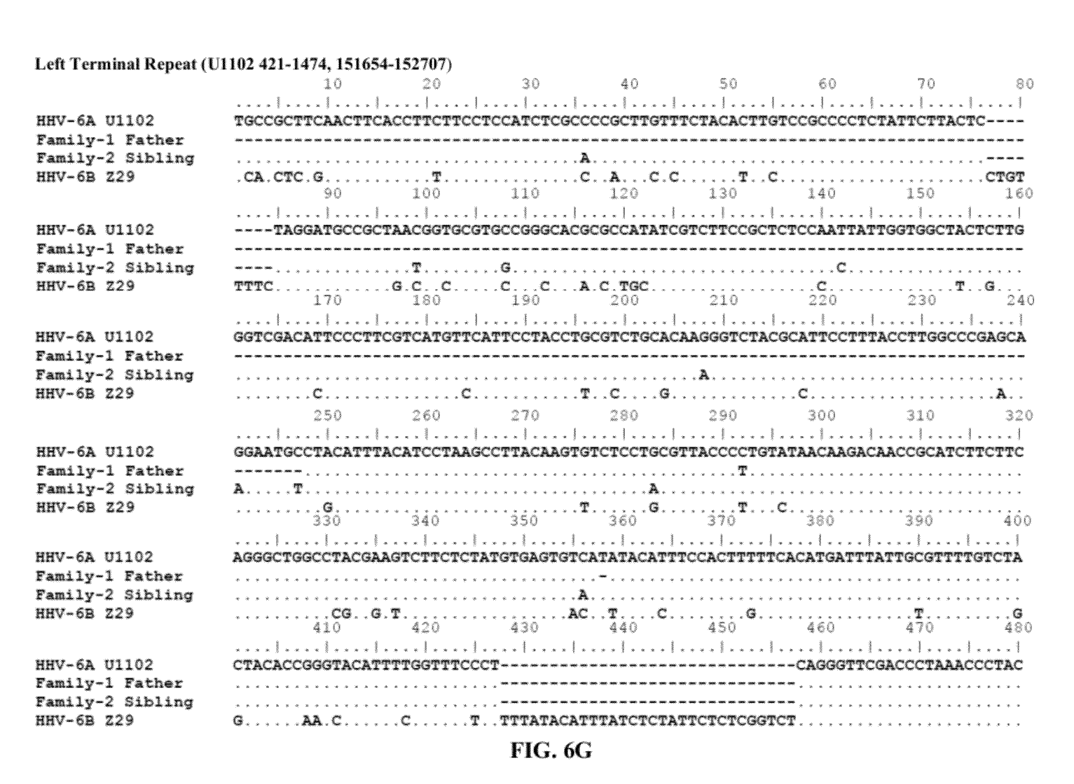

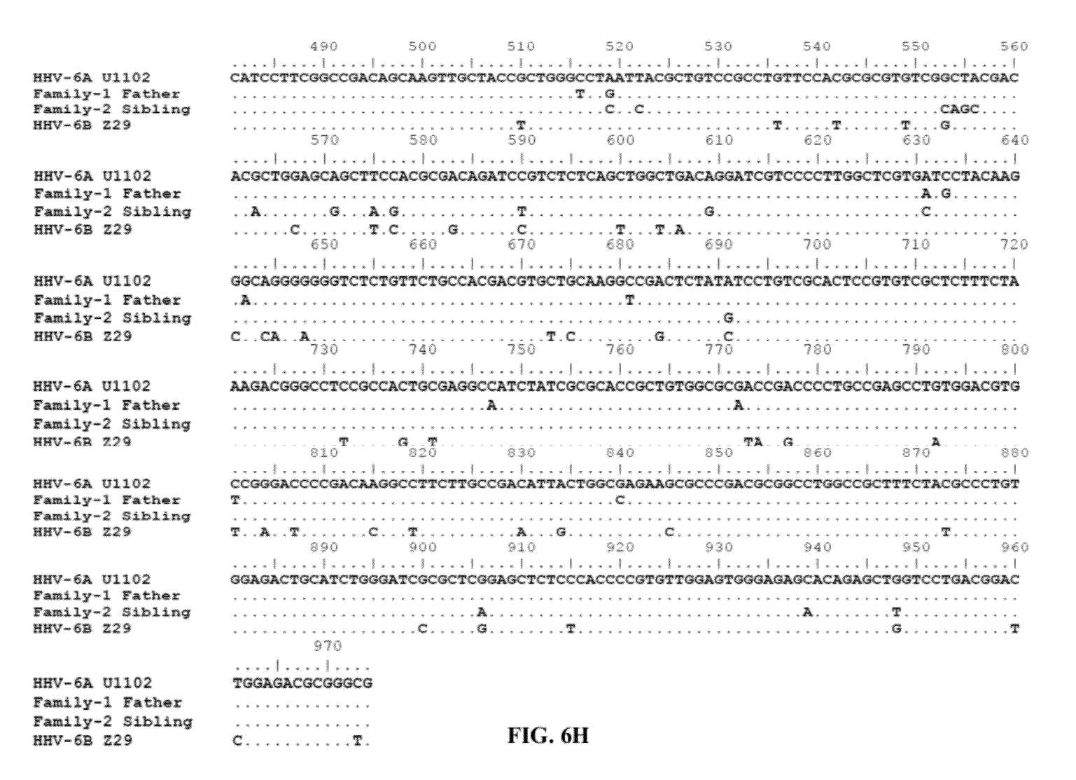

In addition, DNA sequences of amplified open reading frame (ORF) U94 and part of the direct repeat (DR) was sequenced. Viral sequences were identical among members of two families and shared 98% sequence identity with HHV-6A (strain U1102) and only 95% with HHV-6B (Z29) (FIGS. 5-6). Cloning and sequencing of the predominant 1.5 kb amplicon from the DR_{R}-derived primer confirmed the integration of HHV-6 within the telomere of chromosome 17p in the cells of this family (FIG. 2c). The integration site contained 5 TTAGGG repeats, and integration resulted in the loss of 79 nucleotides from the far right end the viral genome.

### Example 5

### In Vitro Integration of HHV-6A into Human T-Cell Line Jjhan and HEK-293 Cells

This Example illustrates methods for in vitro integration of HHV-6 genome into chromosomes. Surprisingly, the chromosome 17p subtelomere-DR_{R} primer pair amplified DNA fragments from DNA isolated from HHV-6A lytically infected Jjhan cells (FIG. 2b), suggesting that integration takes place during productive infection. In this Example, three sets of in vitro experiments were conducted to determine the frequency with which new infection of naïve cell lines with HHV-6A leads to chromosomal integration, and whether chromosomal integration is the sole mechanism by which HHV-6A achieves latency.

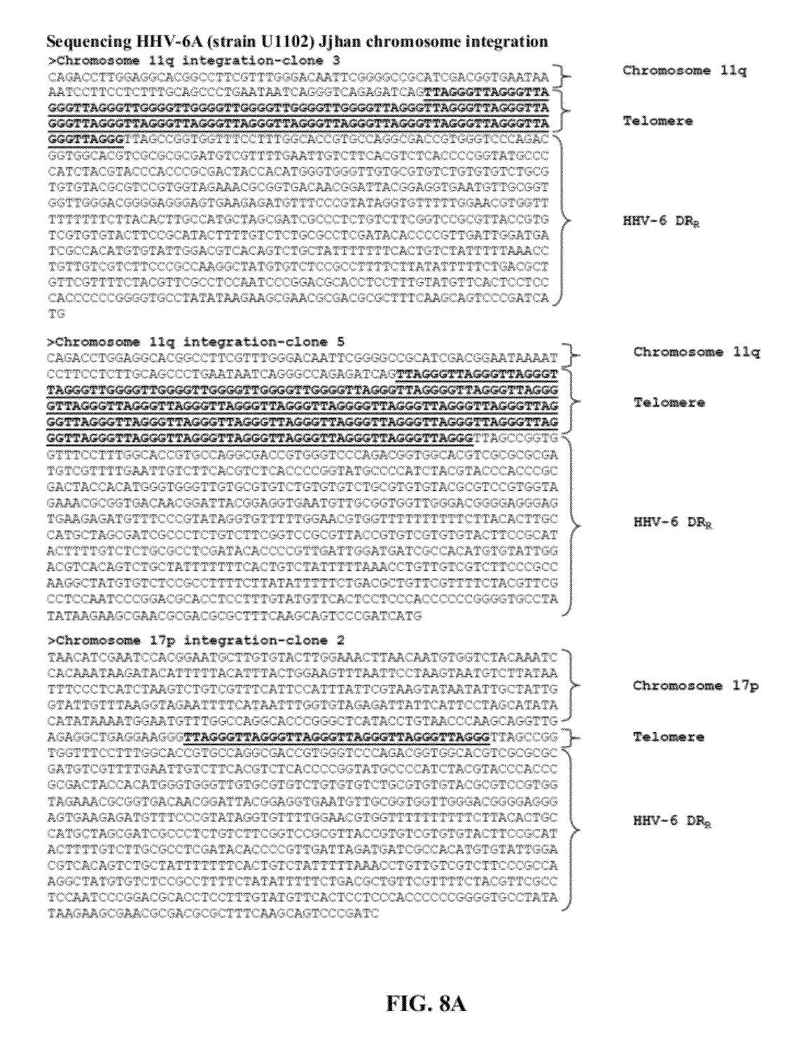

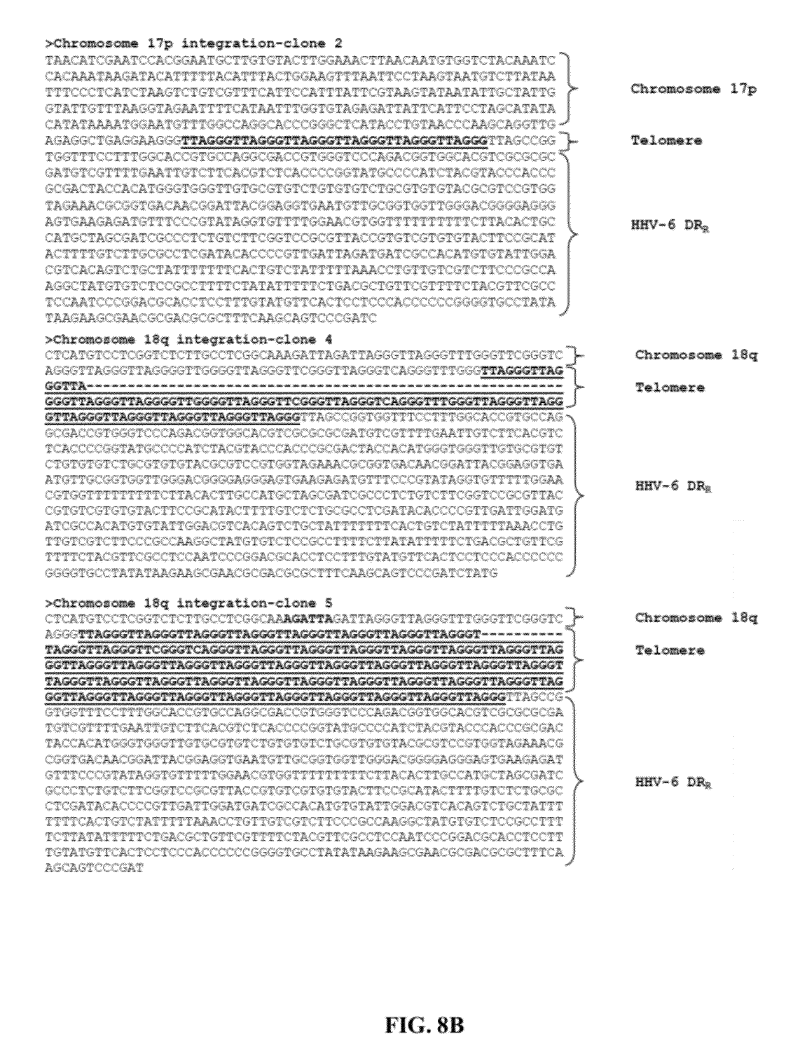

The first set of experiments investigates whether HHV-6A strain U1102 can integrate into telomeres of the T-cell line Jjhan. Jjhan cells are routinely used to propagate HHV-6A, yet the present inventors observed that despite supporting lytic infection, these cells often were not lysed and that many cells survived after the peak of productive infection. The present inventors discovered that in at least some of the infected cells, rather than productive infection leading to lysis, the virus had achieved latency through integration. Specifically, DNA was isolated from cells at the peak of cytopathic effect (CPE), containing 10³ infectious units/ml of virus. The putative viral genome-telomere junction was amplified using subtelomere based primers (11q, 17p, and 18q) (28) and a primer derived from near the right end of the DR_{R}. After cloning and sequencing of PCR products, DNA sequences of virus-chromosome junction were compared with chromosomal DNA sequences and HHV-6A DNA sequences obtained from GenBank for nucleotide sequence analysis. As shown in FIG. 8, the HHV-6A genome is covalently linked with all chromosomes tested, demonstrating that HHV-6A DNA is integrated into the subtelomeric region of the chromosome.

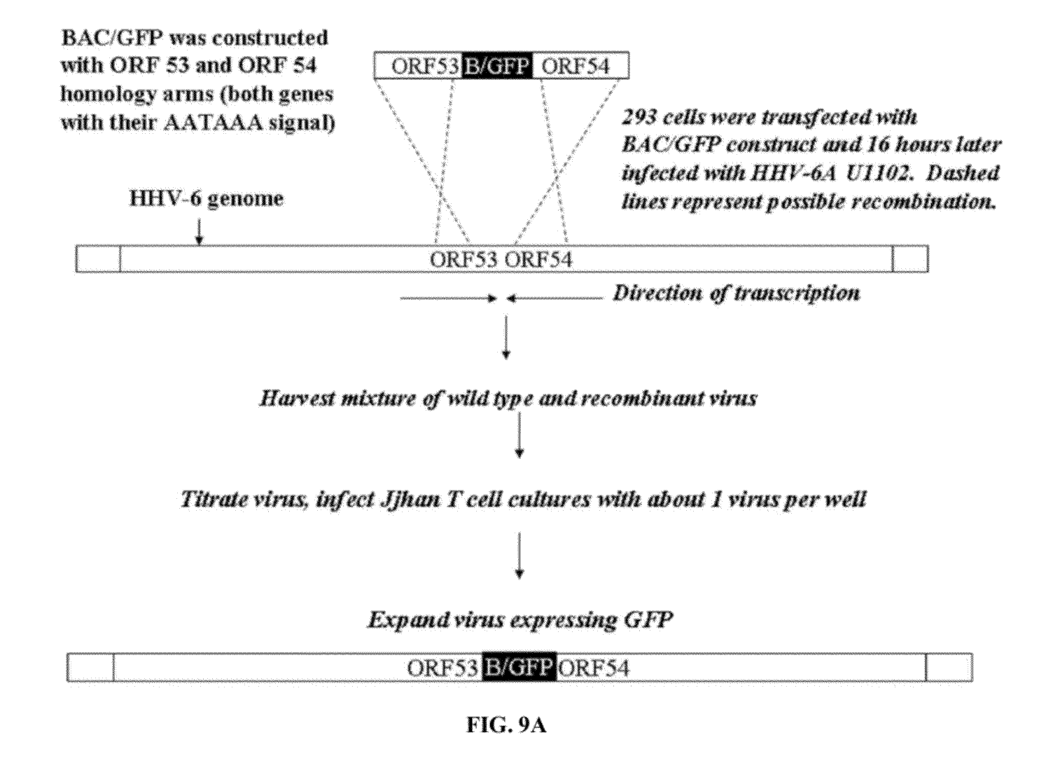

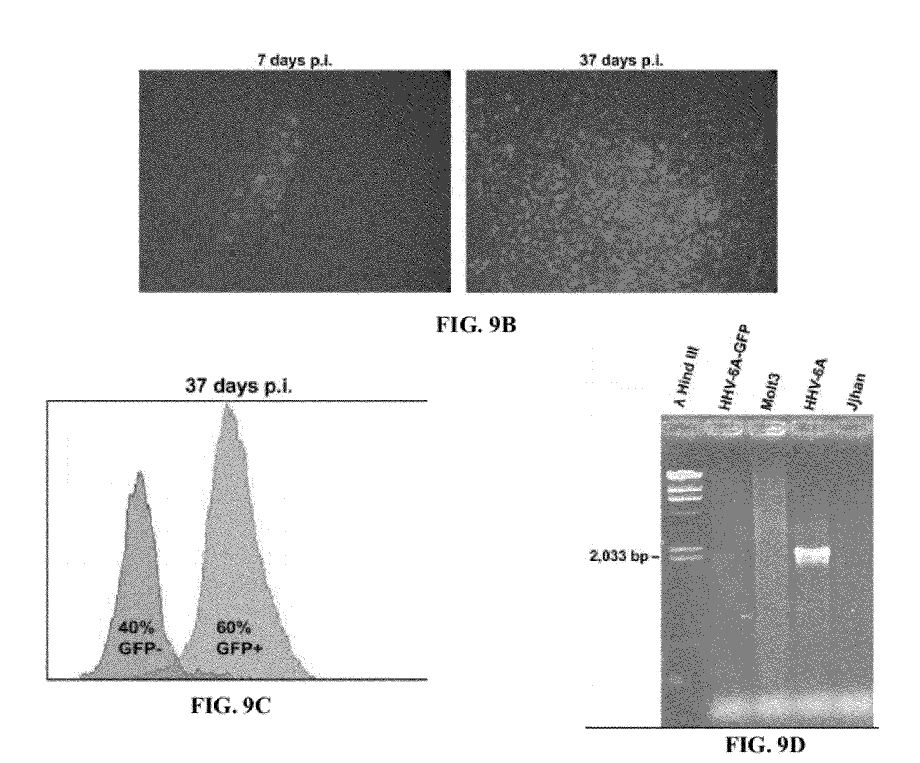

The second set of experiments monitors the progression and spread of infection in these Jjhan cells. HHV-6A recombinant virus (HHV-6A^{GFP}) carrying the green fluorescent protein (GFP) was constructed according to procedures described in FIG. 9a. Specifically, DNA plasmid, in which the GFP expression cassette is flanked by two 2 kb fragments of ORF U53 and ORF U54 cloned from HHV-6A strain U1102, was constructed. Then, several human monolayer cell lines were transfected with the plasmids and screened for cells that supported lytic replication and in which transfection of the plasmid was efficient. A particularly preferred cell line was the human embryonic kidney-293 (HEK-293) cell line (29), as preliminary experiments indicated that HEK-293 cells produce infectious virus at low cell density/confluency. To generate HHV-6A^{G}″ recombinants, a HEK-293 monolayer was then transfected with the ORF U53-GFP-ORF U54 plasmid. The following day, cells were seeded at about 10% confluency and infected with 10³ infectious units of HHV-6A (U1102). After 6 days, characteristic cytopathic effects (e.g., ballooning, refractile giant cells) were observed. The viral titer was determined as 10³ infectious units.

The third set of experiments reveals that the majority of HHV-6 viruses achieve latent infection in host cells, and such latency does not involve the formation of viral episomes. Specifically, the human T-cell line Jjhan was cultured in 96 well plates and each well was infected with about one infectious unit of the potential recombinant virus. In one culture fluorescence microscopy showed a dramatic increase in the number of green fluorescent cells from 7 to 37 days post-infection (FIG. 9b). Only a few very bright fluorescent large multinucleated cells, which were presumably producing infectious virus, were observed. The majority of cells in the well displayed dimmer GFP expression. The distribution of GFP expressing cells is indicated by the fluorescence-activated cell sorting (FACS) analysis. The results, as shown in FIG. 9c, revealed that very few cells displayed bright fluorescence, while 60% of the cells express GFP from 7-37 days post infection.

To evaluate whether fluorescence corresponded to production of HHV-6A^{GFP} virions, two hundred cells of the culture were examined by transmission electron microscopy (TEM) in the Fred Hutchinson Cancer Center EM core laboratory for virus production. The results showed that no virions were observed. The viral DNA was reproducibly detected by PCR, indicating the presence of the viral genome (FIG. 9d). In addition, no free circular or linear viral genomes was determined by the method of Gardella et al. (27). Therefore, the results showed that HHV-6A^{GFP} established primarily latent infection in Jjhan cells cultured for over one month, and that latency did not involve the formation of viral episomes.

### Example 6

### Reactivation of Integrated HHV-6 IN Host Cells

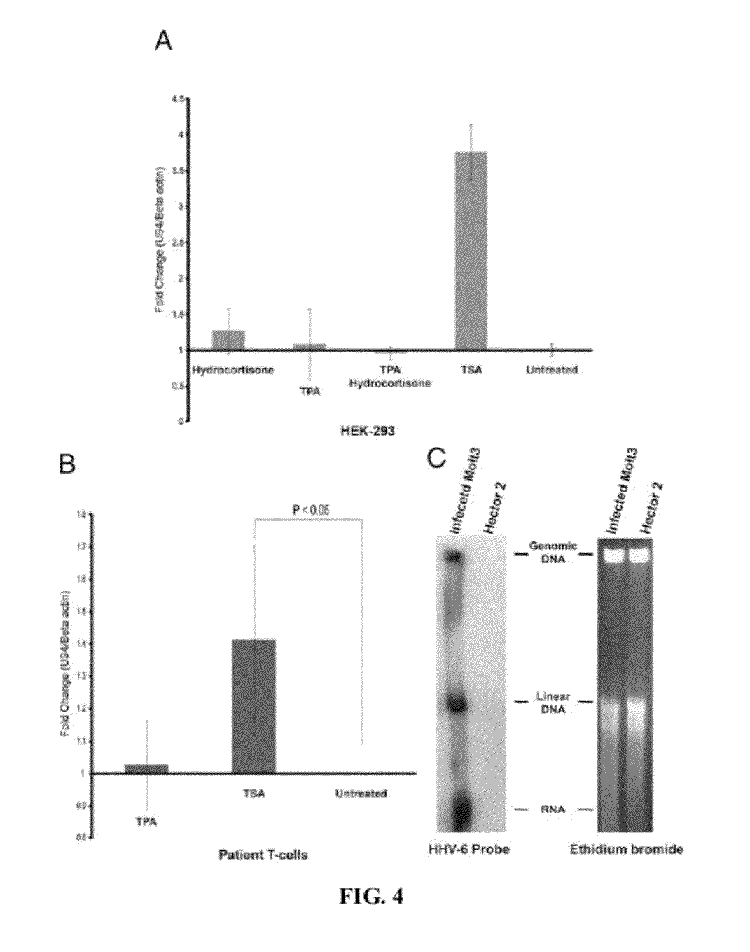

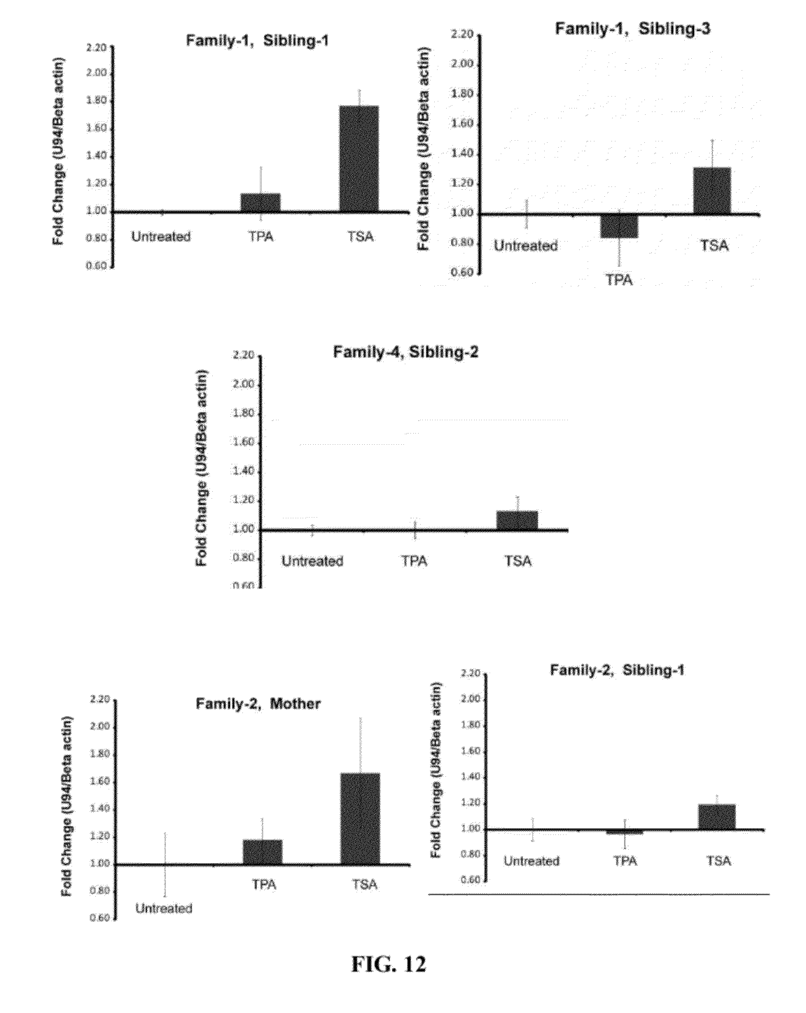

This Example reveals that chromosomally-integrated HHV-6 can reactivate and integration is the sole molecular strategy for achieving viral latency. Briefly, T-cells isolated from five family members and three latently infected HEK-293 cell lines were cultured in AIM-V or DMEM medium supplemented with 10% FCS and treated with known inducers of herpesvirus lytic replication protein kinase-C inducer tetradecanoyl-13 acetate (TPA) (20 ng/ml) and histone deacetylase inhibitor trichostatin-A (TSA) (80 ng/ml) for three days (42, 43). DNA isolated from cells (in triplicate) was subjected to quantitative real time PCR (qPCR) for ORF U94 and fold change ratios of Ct values normalized to beta actin were relative to untreated control. Results of real time PCR (ORF U94) showed a significant increase in the number of copies of viral DNA in TSA-treated cells, relative to untreated cells. Treatment with TPA also causes similar, though milder, reactivation of herpes viruses, compared to that of TSA treatment (FIG. 4ab and FIG. 12).

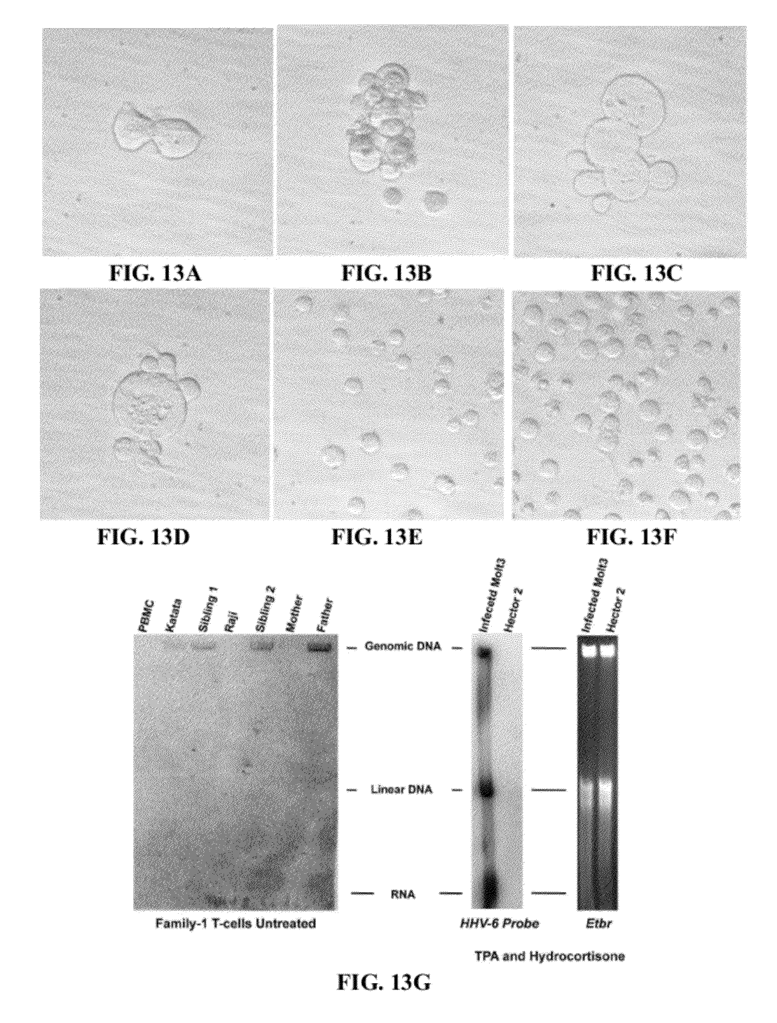

To determine if the increase of viral DNA copy number indicated the production of infectious virus, PBMCs from six members of Family-1 and Family-2 were isolated. The PBMCs were cultured in the presence of TPA and hydrocortisone, which resulted in a marked increase in copy number. The PBMC cells were also co-cultured with Molt3 cells in the presence of TPA and hydrocortisone. Syncytia were formed in the Molt3 cells infected with the virus obtained from TPA- or hydrocortisone-induced T cells, and replicating linear viral DNA and RNA were detected in these Molt3 cells by Gardella gel (FIGS. 4c and 13). Sequencing of the viral DNA in the Molt3 cells revealed that the viral DNA was identical to the integrated sequence in the cells of Family-1 and Family-2 (FIG. 6).

### Example 7

### Construction of Plasmids and Recombinant Virus

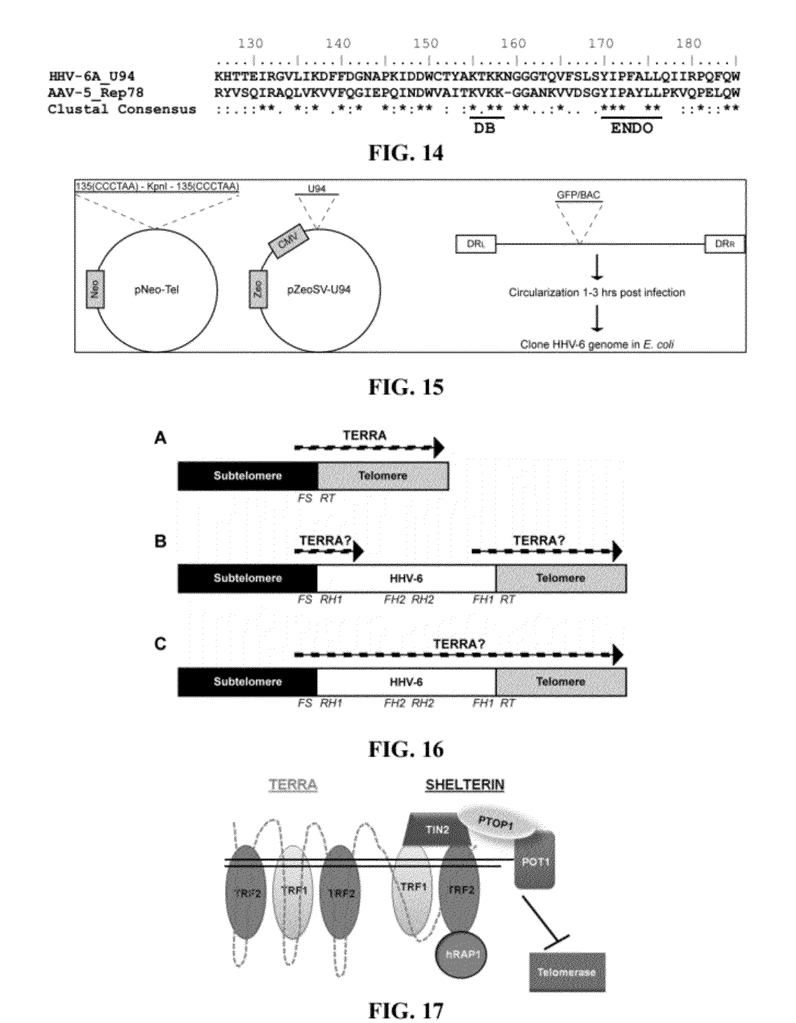

This Example illustrates construction of plasmids carrying HHV-6 U94 rep gene and recombinant HHV-6 virus. HHV-6 U94 rep gene has 24% amino acid identity with various serotypes of the adeno-associated virus (AAV) Rep78 protein (Thomson et al., 1991, see Appendix for alignment of the proteins), which is required for integration and replication of AAV viral genome (reviewed by Burns and Parrish, Fields Virology). The crystal structure of the AAV-5 endonuclease domain has been determined (Burgess Hickman et al., 2002). One of the two Tyr residues in the endonuclease active site of AAV-2 is present in the HHV-6A U94/rep sequence, and the DNA binding motif of the AAV-2 protein is present in the U94/rep sequence (FIG. 14).

In addition, the functions of U94/rep and AAV Rep78 are similar as evidenced by complementation experiments, which demonstrated that U94/rep can reconstitute DNA replication of a Rep78-deficient AAV-2 virus (Thomson et al., 1994). Nuclear localization and gene expression characteristics of U94/rep are consistent with its role as an integrase (Mori et al., 2000). The U94/rep protein forms punctuate complexes in the nucleus. Several size classes of U94/rep transcripts have been described and their temporal expression is complex. Some mRNAs are expressed under immediate early conditions (Mirandola et al., 1998, Mori et al. 2000). U94/rep is also expressed during latency. Importantly, U94/rep inhibits HHV-6 lytic replication (Caselli et al., 2006), suggesting that this gene is “pushing” HHV-6 towards latency/integration.

Briefly, two plasmids are constructed. The first plasmid contains a selectable marker neo/G418 flanked by two blocks of TTAGGG repeats (FIG. 15, left panel). The plasmid has a unique KpnI restriction site, which is located between the telomere repeats and linearizes the integrating plasmid. The direction of Neo transcription is the same as that of TTAGGG repeats (to avoid possible TERRA antisense effects). The second plasmid, pZeoSV-U94, encodes the HHV-6A U94/rep open reading frame cloned in the expression vector pZeoSV controlled by the SV40 large T promoter. This plasmid is used for determining whether U94/rep enhances telomere-specific recombination.

In addition, a recombinant replication-competent HHV-6A virus (FIG. 15, right panel) (Arbuckle et al., 2010) is constructed. This recombinant virus encodes GFP and BAC vector elements, which are required for cloning of the entire HHV-6A genome in E. coli (FIG. 15). This recombinant HHV-6A virus is used for generating replication-competent HHV-6A in E. coli.

### Example 8

### Identification of U94/Rep Gene as Viral Gene Associated with Telomere-Specific Integration

To determine whether U94/rep enhances telomere-specific recombination, U94/rep expression vector and the telomere-targeting vector (FIG. 15) are transfected into HEK-293 cells. As demonstrated in Example 5, these cells can be readily transfected by HHV-6A viruses, which integrate into host telomeres. After transfection and G418 antibiotic selection, single clones are selected and the frequency of integration events is evaluated by qPCR and FISH, and compared to control cells. Control cells are transfected only with pNeo-Tel and empty pZeoSV expression vector.

The in vitro transfection assays will reveal that U94/rep alone promotes telomere-specific integration and would identify functional domains of the U94/rep gene. These assays will also determine if U94/rep enhances telomere-specific integration in the absence of other viral genes.

### Example 9

### Identification of U94/Rep as Telomere-Specific Binding Protein

The Example investigates whether U94/rep is a telomere-specific binding protein. The initial assay is based on constructing a C and N terminus epitope-tagged (Flag, His-tag) recombinant U94/rep vector. After transfection, cell extracts will be incubated with radiolabeled single or double stranded TTAGGG repeats of varying length. Controls will be randomized oligonucleotides consisting of the same bases present in the specific telomere repeats. Protein-DNA complexes will be visualized by “bandshift” assays, as previously described by the present inventors (Geck et al., 1994). Competition assays with cold DNA and quantitative analysis by scanning radioactive gels will also be performed. Large scale filter binding assays will be performed using the strategy developed for AAV studies (Owens et al., 1993).

Nuclease assays are performed using supercoiled plasmids containing telomere repeats incubated with purified recombinant U94/rep. Conformational change of the plasmid is evaluated quantitatively by separating various forms of plasmids (supercoiled, nicked, linearized) by agarose gel electrophoresis and Southern blotting with ³²P labeled plasmid probes. The autoradiographs are quantitatively evaluated by beta scanning of the Southern blot filters.

The DNA binding assay results can reveal that U94/rep is a telomere-specific binding protein and it cleaves telomeres specifically. The DNA binding motif of AAV-5 Rep78 and U94/rep show strong conservation (FIG. 14), and U94/rep binds single-stranded DNA (Dhepakson et al. 2002). The DNA binding assays also reveal whether U94/rep is a TTAGGG repeat-specific binding protein.

### Example 10

### Production of Recombinant HHV-6 Virus in E. Coli

In this Example, a replication-competent recombinant HHV-6A virus is cloned in E. coli to produce sufficient quantities of GFP/BAC-HHV-6A. The T cell line Jjhan is infected with this virus stock, and 2 hours after infection, pre-replication circularized DNA is isolated and cloned in E. coli (Delecluse et al., 1998, Collins et al., 2002).

The BAC construct is used to generate mutations in U94/rep and in the viral telomere repeats using standard protocols (Muyrers J P, et al. 1999). Controls include a virus with a marker-rescued U94 gene. To examine the effects of deletion of U94 sequences on viral gene expression, stop codons and frame shift mutations are engineered into the BAC clone near the initiation codon to eliminate U94/rep expression. GFP-expressing control and U94/rep infected cells are cultured, GFP positive cells are sorted by FACS, and FISH experiments are conducted to compare telomere-specific integration of the control versus mutant-infected cells. Mutagenesis on the DNA binding motif and the nuclease domain (see amino acids underlined/bracketed in FIG. 14) is performed.

Due to the similarities between AAV and HHV-6A Rep proteins and other features of the HHV-6A U94 gene, the results show that the HHV-6A protein plays a significant role in telomere-specific integration. Mutagenesis of the virus reveals whether U94/rep is involved in integration. It is postulated that U94/rep plays a role in telomere-specific integration.

### Example 11

### Inhibition of HHV-6 U94/Rep Gene

In this Example, the U94/rep gene is silenced by siRNA and the frequency of viral genome integration is compared to controls using qPCR and FISH. The effect of the siRNA constructs is tested in a T cell line, Jjhan, which produces ˜1000 infectious viruses/ml. HHV-6A also integrates into Jjhan cells (Arbuckle et al., 2010). The cells are infected with GFP-HHV-6A and treated with siRNA constructs or control constructs (random siRNA) after 1 week at various intervals (intervals will be determined after pilot studies). The ratio of bright GFP expressing cells, representing replicating virus, to dim, latently-infected control cells and siRNA-treated cells is compared by FACS at various time points after siRNA treatments. The effect of siRNA is also evaluated by determining the number of infectious units by limiting dilution and by Gardella type agarose gels that can distinguish lytic and latent virus replication (Gardella et al., 1984, Arbuckle et al., 2010).

The results show that inhibition of U94/rep enhances lytic replication since U94/rep suppresses productive infection (Caselli et al., 2006). The results also show that inhibition of U94/rep reduces frequency of integration of HHV-6 into host chromosome.

### Example 12

### Determination of Modulation of Terra Expression by Telomere Specific HHV-6-Integration

This Example investigates the effects of HHV-6 genome integration on expression of telomere-specific nuclear transcripts TERRA. TERRA promoters are located in the subtelomeres, and the transcripts span across large portions of telomeres (FIG. 16, top). TERRA is essential for the maintenance of telomere integrity (Azzalin et al., 2007, Luke and Lingner, 2009). TERRA stabilizes the shelterin telomere-protein complex and facilitates telomere heterochromatin formation through direct interaction with TRF1 and TRF2 proteins (de Lange, 2005) (FIGS. 16 and 17). Telomere length in somatic cells is 5-15 kb, and, despite the shelterin complex, 250-300 bp are lost from the end of telomeres after every cell division. When telomeres reach a critical minimal length, cells undergo replicative senescence and/or apoptosis (Reithman, 2008).

FIG. 17 shows that telomeres form a complex with 6 proteins that protect the ends of chromosomes from double-stranded breaks and chromosome fusions (de Lange, 2005, Raynaud et al., 2008). TRF1 (TTAGGG Repeat Factor 1), TRF2 (TTAGGG Repeat Factor 2), and Pot1 (Protection of Telomeres 1) directly bind to the telomeric TTAGGG repeat, while the shelterin complex is maintained through TIN2 (TRF1-Interacting Nuclear Factor 2), TPP1 (TINT1, PIP1, PYOP1) and RAP1 (Repressor Activator Protein 1).

There is a connection between telomere binding proteins and Epstein-Barr virus (EBV) latent replication. TRF2 binds to the EBV-encoded TTAGGGTTA repeat (imperfect TTAGGG repeat) within the origin of plasmid replication (OriP) in cooperation with viral latency gene EBNA-1 (Deng et al., 2002, Zhou et al., 2009). TRF2 and EBNA-1 binding of OriP stabilizes the EBV episome during latency and enables the non-covalent attachment to metaphase chromosomes; this process ensures division of the viral genome among daughter cells (Marechal et al., 1999, Zhou et al., 2009). Additionally, Lieberman and colleagues found that EBNA-1 directly interacted with TERRA; however, the purpose of this interaction has not been fully elucidated (Deng et al., 2009).

It is postulated that after integration, TERRA expression is interrupted by the insertion of the 160 kb genome of HHV-6A. It is postulated that (1) there is a TERRA-like promoter in the HHV-6A genome (FIG. 16, center model); or (2) TERRA transcription is initiated in the subtelomere and is elongated across the HHV-6A genome (FIG. 16, bottom model) to explain stable maintenance of the HHV-6A-invaded-telomere.

To map the three possible species of TERRA, nuclear polyadenylated RNA is isolated and several RT-PCR reactions are performed (adapted from Caslini et al., 2009). The telomere-specific primer complementary to TERRA (RT) is composed of four repetitions of CCCTAA (depicted below the grey telomere regions, FIG. 16). Forward primers are derived from the subtelomere of chromosome 17 and 18 (FS), as we previously described (Arbuckle et al., 2010). The expression of RNA across the viral genome is also evaluated using the forward and reverse primers depicted in FIG. 16 (only one pair is shown). In addition, the PCR reaction avoids amplification of viral genome regions that are expressed during latency (Mirandola et al., 1998).

The RT-PCR experiments provide preliminary evidence of TERRA expression according to various possible scenarios (FIGS. 16A, B, and C). The RT-PCR results are confirmed by Northern blot experiments. Finally, if the data indicate the existence of an HHV-6A TERRA primer, its promoter is mapped by primer extension and 5′ RACE, and by other standard methods we used previously (Geck et al., 1994).

This Example also reveals whether TERRA interacts with the chromosomal end occupied by the integrated HHV-6A. The experiments are performed in a procedure similar to DNA-FISH, as is previously described by the present inventors (Arbuckle et al., 2010), except that a cy5-conjugated UUAGGG probe is used to detect TERRA, a FITC-conjugated HHV-6A pMF311-2 and pMF335-6 cosmid probe (Neipel et al., 1991), and DAPI staining of metaphase chromosomes. Co-hybridization with all three probes confirms the direct interaction between HHV-6A and TERRA. As a control, HHV-6A infected cells are treated with RNase A to abolish the TERRA signal and confirm that the UUAGGG probe detects RNA (TERRA) and not single-stranded telomeric DNA located at the end of chromosome telomeres (Azzalin et al., 2007). To enhance sensitivity, TERRA can be affinity-selected using a TAACCC telomere-complementary oligonucleotide affinity column. Also, oligo dT selection of polyadenylated transcripts can enhance the level of detection. Alternatively, RNA-FISH can be used for detection of TERRA. As the present inventors have identified HHV-6A integration into telomeres through DNA-FISH (Arbuckle et al., 2010), the binding of TERRA to HHV-6A genome can also be detected through RNA-FISH.

The results show that TERRA transcripts are expressed in the HHV-6A-occupied telomere, as shown in FIG. 16B, since the subtelomere TERRA promoter remains present. TERRA expression is critical for maintaining telomere integrity. It is also postulated that HHV-6A encodes an as-yet unrecognized TERRA promoter. A less likely possibility is that TERRA is transcribed across the entire 160 kb HHV-6A genome (FIG. 16C), since latent transcripts have been described and mapped to very limited regions of the viral genome (Mirandola et al., 1998, Mori et al., 2000).

### Example 13

### Determination of Modulation of the Shelterin Complex by Telomere Specific HHV-6-Integration

This Example investigates whether telomere-specific HHV-6 integration alters the shelterin complex. The integration of HHV-6A may transiently or permanently alter shelterin—the protein complex that interacts with telomeres. The association of telomere-specific protein with regions near the viral genome is examined in cell lines that carry integrated HHV-6A (Arbuckle et al., 2010).

It is postulated the U94/rep protein is required for telomere-specific integration. One possible mechanism by which U94/rep facilitates this integration is by forming a bond between the viral TTAGGG repeats and telomere binding proteins. In this Example, tagged U94/rep is overexpressed in T-cells and HEK-293 cells. The U94 protein and associated shelterin proteins are immunoprecipitated and analyzed by Western blotting using various antibodies provided by Drs. de Lange and Henderson.

To determine whether telomere proteins TRF1, TRF2, hRAP1 and POT1 bind to TTAGGG viral repeat, chromatin immunoprecipitation (ChIP) is performed on cells with lytic or latent integrated HHV-6A. HHV-6A virus inoculum is used to infect Jjhan cells (in triplicate). Next, stages of lytic and latent infection are monitored through visual observation of cytopathic effects and analyzed for the presence and/or absence of replication linear DNA through Southern hybridization of Gardella gel with ³²P-labeled HHV-6A cosmid probe (Arbuckle et al., 2010). ChIP with TRF1, TRF2, hRAP1, and POT1 is also performed using cell lines established in our laboratory (Arbuckle et al., 2010). These cell lines contain integrated HHV-6A and were established from in vivo-integrated peripheral blood mononuclear T cells from patients, and in vitro-integrated HEK-293 cells.

Cells are subjected to formaldehyde cross-linking, immunoprecipitation with anti-TRF1, anti-TRF2, anti-hRAP1, anti-POT1, and anti-GFP IgG (negative control) antibodies and identification of associated DNA by PCR. Quantitative real-time PCR (qPCR) using Sybr green chemistry is used to identify the DNA fragment associated with shelterin proteins. Primers are designed to amplify a 150-200 bp amplicon located within 300 bp of the virus encoded TTAGGG repeat will be used to record telomere protein binding.

It is possible that ORF U94 acts in association with other viral proteins to allow interaction with shelterin proteins. Thus, cells are transfected with the U94/rep recombinant construct and infected with virus; protein-protein interaction assays are then performed. In addition, since the HHV-6A titer is low, it may be difficult to PCR-amplify associated DNA fragments following ChIP. To address this potential problem, HHV-6A^{GFP} virus (Arbuckle et al., 2010) can be employed. GFP expressing cells are isolated through FACS, thereby increasing the number of cells infected with virus.

The results show that U94/rep protein interacts with protein(s) of the shelterin complex. Since telomeres are protected by the shelterin protein complex, HHV-6A must have evolved a specific process to promote homologous recombination between the viral and cellular TTAGGG repeats, as TRF1, TRF2 and hRAP1 bind to imperfect telomere repeat TTAGGGTTA in the OriP during latent EBV infection (Deng et al., 2002). This example reveals the dynamics of the telomere shelterin complex binding to the TTAGGG repeat in the DR_{L} and DR_{R} of HHV-6A. This binding plays a critical role in virus integration and protects the TTAGGG repeat region of the HHV-6A genome.

### Example 14

### Determination of the Effect of HHV-6 Integration on Length of Telomeres

The present inventors have observed that stable integration of HHV-6 is semi-random; about 66% of reported integration sites in families and individuals are found in three specific chromosomal ends (1q44, 17p13.3 and 22q13)., and some latently infected cells grow at a slower rate than uninfected cells, and they may stop growing. Integrated HHV-6 has been detected in “preferential” integration sites (Table 2). This finding can be explained by two alternative mechanisms. First, certain chromosome ends may be more accessible for homologous recombination between the viral and chromosomal TTAGGG repeats. Alternatively, HHV-6 may integrate randomly into all 46 chromosome ends, but the integrated genome may be unstable in some telomeres. After a series of divisions, cells with unstable chromosomes are “negatively” selected.

TABLE 2

| Integr. Site | #Families | #Individuals | References |
|---|---|---|---|
| 1q44 | 4 | 7 | (2, 3, 9) |
| 9q34.3 | 2 | 2 | (6) |
| 10q26 | 1 | 1 | (6) |
| 11p15.5 | 1 | 1 | (6) |
| 17p13.3 | 7 | 9 | (1, 5, 6) |
| 19q13.4 | 2 | 2 | (6) |
| 18q23 | 1 | 3 | (1) |
| 22q13 | 7 | 9 | |

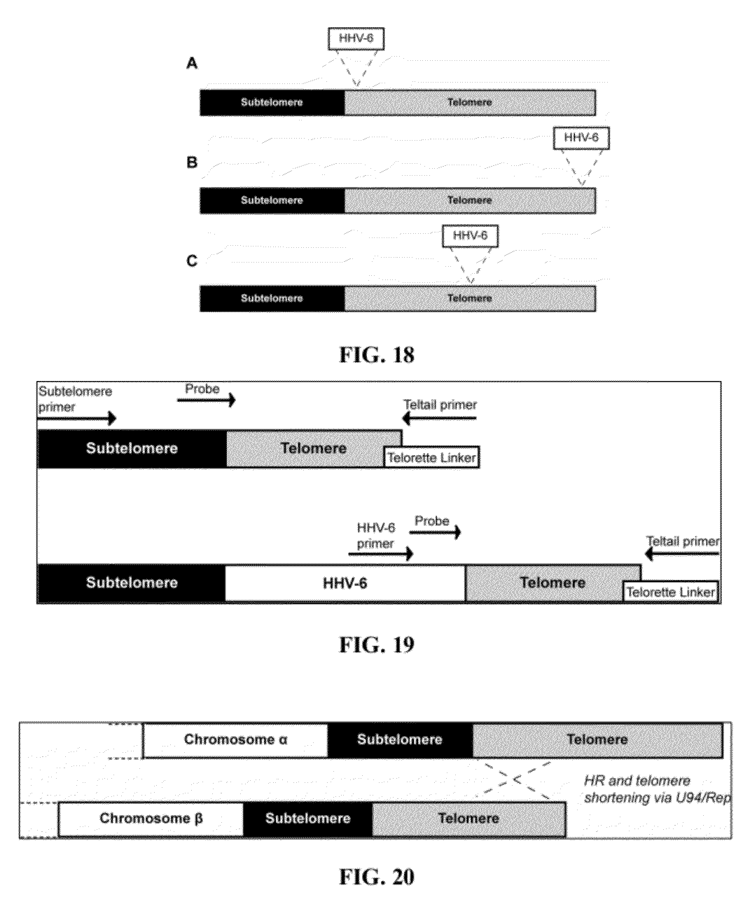

The mechanisms through which HHV-6 integration causes telomere instability are postulated: first, as illustrated in FIG. 18, stability of the telomere is dependent upon the relative integration site; second, virus infection may promote recombination between chromosomes, leading to chromosome instability (FIG. 20). This possibility is supported by preliminary data showing that the viral genome insertion occurs by homologous recombination between the cellular and viral TTAGGG repeats. Third, the frequency of reactivation of latent HHV-6 may depend upon which chromosomal end the virus integrates into.

In addition, the present inventors established an in vitro system to study integration of HHV-6A in cloned cell lines (Arbuckle et al., 2010). HEK-293 cells were infected with HHV-6A, and 27 single cells were cloned and expanded in 96-well plates. During the cloning process we noted significant heterogeneity in single cell clone behavior. Of the 27 clones, 13 reached confluency within 10-12 days; 12 clones reached confluency more slowly, requiring 16-18 days. These slowly growing clones were expanded and analyzed for HHV-6A by PCR. Of 12 clones, six clones (50%) were PCR positive. After further subculture, only 3 remained consistently HHV-6A positive as tested by PCR and all cells tested by FISH contained integrated HHV-6A as described (Arbuckle et al, 2010). The other 3 clones that were initially positive have “lost” the viral genome after subculturing. All HHV-6A-containing clones were negative for lytic HHV-6A replication (tested by the method of Gardella). 2 clones formed a small colony of about 30-100 cells. The colonies persisted for >2 weeks but never expanded. These clones were not tested for HHV-6A due to the small numbers of cells.

These data show that the fate of cells after latent infection varies greatly, and this variability is likely dependent upon the relative position of integration into telomeres or the specific chromosomal end. By sequencing HHV-6A and HHV-6B integration sites, we determined that the right end (DR_{R}) of the viral genome is oriented towards the subtelomere. The bulk of the cellular telomere repeats are joined at the left end (DR_{L}) of the viral genome (Arbuckle et al., 2010). To determine if HHV-6 had integrated into the family members' chromosomes, as described above we performed fluorescence in situ hybridization (FISH) in T-cell cultures derived from peripheral blood. The experiments were performed by two independent laboratories (Children's Cancer Research Institute, Vienna, Austria and University of Minnesota, Minneapolis, Minn.). Each laboratory was unaware of which individual or family the specimens were obtained from. In each experiment, HHV-6-specific fluorescence was detected in association with the telomeric regions of chromosomes. The specific chromosome was identified by co-hybridization with viral and chromosome-specific probes. HHV-6-specific FISH signal was detected in chromosomes 17p13.3, 18q23 and 22q, respectively, in the 3 families studied with FISH. Furthermore, HHV-6 and telomere FISH signals overlapped. Specific telomeres will be determined.

This Example investigates the length and fate of telomeres after integration of HHV-6 viral genome into replicating cells at various time points. The sequencing data show that HHV-6A can stably integrate into telomeres if the viral genome is near the subtelomere (FIG. 18). However telomeres may become unstable if HHV-6A integrates near the end of the chromosome (FIG. 18B). Similarly, if the HHV-6A genome integration “splits” the telomere (FIG. 18C), the sudden loss of effective telomere length, may trigger chromosome instability.

In this Example, telomere length of normal and virus-invaded chromosomes will be measured over time. It is postulated that the viral genome can integrate at various positions in telomeres as shown in FIG. 18). The single telomere length analysis (STELA) of Baird et al. (2003) can be performed for this analysis (FIG. 19).

The first step of the STELA assay (FIG. 19) is ligation of an oligonucleotide, called Telorette, to the end of the telomere. The Telorette linker is complementary to the top 3′ single strand G-rich telomere overhang. The 5′ end of the Telorette sequence is unique and allows amplification of the entire telomere region. The Telltale primer, complementary to the Telorette, and either the primer in the subtelomere or in the left end of the HHV-6A genome, is used to amplify the telomeres of the normal or HHV-6A-occupied chromosome allele. The relative size of the amplified products is identified by Southern blotting using probes derived from either the subtelomere or from the left end of HHV-6A.

The results reveal a direct correlation between the integration site and the loss of telomere, since telomere length is essential for chromosome stability. It is postulated that stable HHV-6A positive clones arise when the viral genome is located near the subtelomere.

### Example 15

### Determination of Preferential Telomeric Site of HHV-6 Integration

This Example investigates whether HHV-6A integrates preferentially in a subset of telomeres. Integration sites are identified shortly after infection, and are compared with integration sites that have undergone a series of replication cycles. This approach is based on analysis of metaphase chromosomes from latently infected cells by FACS, and chromosomes are sorted according to chromosome size. Various size classes of chromosomes are tested for HHV-6A and unique chromosome markers by quantitative PCR. Chromosome sorting according to size is an established method and has been used to estimate the length of telomeres in individual chromosomes (Ng and Carter, 2006).

The first goal of these experiments is to determine if “preferential” integration occurs shortly after infection. Normal PHA-activated peripheral blood T cells from healthy donors cultured in IL-2-containing medium are infected with GFP-expressing HHV-6A (Arbuckle et al., 2010). T cells are cultured in IL-2 medium and tested for GFP expression (Arbuckle et al., 2010). “Dim” GFP-expressing latently-infected cells are selected and further cultured. Chromosomes, including metaphase chromosomes generated at various time points after infection (e.g., weekly), are sorted according to size. DNA is prepared from chromosome fractions and the amount of latent HHV-6A is determined by qPCR. Specific chromosomes are identified in the fractions by PCR analysis of chromosome-specific genes.

The results reveal that shortly after infection, HHV-6A can integrate into most—if not all—chromosomes. However, based on the biased distribution of integration sites (Table 2), fewer integration sites are identified after a series of cell divisions have been completed.

### Example 16

### Identification of Deletion of Telomere Genes During HHV-6 Integration

This Example investigates whether integration of HHV-6 genome DNA results in deletion of host telomere genes, using FISH technology. FISH is suitable for determining the specific integration site and for evaluating chromosomes for telomere loss as described (Martens et al., 1998). FIG. 19 depicts a possible scenario whereby HHV-6A infection promotes telomere-telomere recombination, leading to loss of telomeres in some chromosomes. Cells are infected with green fluorescent protein (GFP)-expressing HHV-6A, then GFP expressing cells are collected and viral integration sites are determined by FISH analysis using viral and chromosome specific probes. In addition, the viral DNA-containing and normal chromosome allele are compared in the cloned latent HHV-6A-carrying cell lines, and induction of virus replication and the extent of virus replication are monitored.

The results show that some clones stably maintain the integrated viral genome; while other clones may show loss of telomeres and viral genome, ultimately causing severe loss of the end of the chromosome that has been destabilized by the integrated viral genome.

### Example 17

### Determination of Chromosomal-Specific Viral Reactivation

This Example investigates whether the frequency of HHV-6 reactivation in host cells depends on the chromosome and telomeric site of viral integration. Briefly, various inducers of herpesvirus reactivation are used to compare reactivation frequency from various telomeres as described (Arbuckle et al., 2010). New and existing latently infected cloned cell lines are used for these experiments and reactivation is measured as described (Arbuckle et al., 2010). In addition, developed qPCR and Gardella gel-based methods illustrated in the Examples are employed (Arbuckle et al., 2010). The results identify chromosomes and telomeres that are more transcriptionally active, and thus, result in more frequent HHV-6 reactivation.

All references, including publications, patent applications and patents, cited herein are hereby incorporated by reference to the same extent as if each reference was individually and specifically indicated to be incorporated by reference and was set forth in its entirety herein.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context). It should be understood that the examples and embodiments described herein are for illustrative purposes only and that various modifications or changes in light thereof will be suggested to persons skilled in the art and are to be included within the spirit and purview of this application.

### REFERENCES

1. Salahuddin S Z, et al. (1986) Isolation of a new virus, HBLV, in patients with lymphoproliferative disorders. (Translated from eng) Science (New York, N.Y. 234(4776):596-601 (in eng).
2. Dominguez G, et al. (1999) Human herpesvirus 6B genome sequence: coding content and comparison with human herpesvirus 6A. Journal of virology 73(10):8040-8052.
3. Gompels U A, et al. (1995) The DNA sequence of human herpesvirus-6: structure, coding content, and genome evolution. Virology 209(1):29-51.
4. Yamanishi K, et al. (1988) Identification of human herpesvirus-6 as a causal agent for exanthem subitum. Lancet 1(8594):1065-1067.
5. Ablashi D. et al. (1993) Human herpesvirus-6 strain groups: a nomenclature Arch. Virol. 129: 363-366
6. Asano Y, et al. (1994) Clinical features of infants with primary human herpesvirus 6 infection (exanthem subitum, roseola infantum). Pediatrics 93(1):104-108.
7. Lusso P, et al. (1991) Productive infection of CD4+ and CD8+ mature human T cell populations and clones by human herpesvirus 6. Transcriptional down-regulation of CD3. Immunol 147(2):685-691.
8. Takahashi K, et al. (1989) Predominant CD4 T-lymphocyte tropism of human herpesvirus 6-related virus. (Translated from eng) Journal of virology 63(7):3161-3163 (in eng).
9. De Bolle L, Naesens L, & De Clercq E (2005) Update on human herpesvirus 6 biology, clinical features, and therapy. Clin Microbiol Rev 18(1):217-245.
10. Dockrell D H & Paya C V (2001) Human herpesvirus-6 and -7 in transplantation. (Translated from eng) Rev Med Virol 11(1):23-36 (in eng).
11. Jones C M, Dunn H G, Thomas E E, Cone R W, & Weber J M (1994) Acute encephalopathy and status epilepticus associated with human herpes virus 6 infection. Dev Med Child Neurol 36(7):646-650.
12. Donati D, et al. (2003) Detection of human herpesvirus-6 in mesial temporal lobe epilepsy surgical brain resections. (Translated from eng) Neurology 61(10):1405-1411 (in eng).
13. Yamashita N & Morishima T (2005) HHV-6 and seizures. (Translated from eng) Herpes 12(2):46-49 (in eng).
14. Lusso P, et al. (2007) Human herpesvirus 6A accelerates AIDS progression in macaques. (Translated from eng) Proceedings of the National Academy of Sciences of the United States of America 104(12):5067-5072 (in eng).
15. Lusso P, et al. (1989) Productive dual infection of human CD4+ T lymphocytes by HIV-1 and HHV-6. (Translated from eng) Nature 337(6205):370-373 (in eng).
16. Cermelli C, et al. (2003) High frequency of human herpesvirus 6 DNA in multiple sclerosis plaques isolated by laser microdissection. J Infect Dis 187(9):1377-1387.
17. Goodman A D, Mock D J, Powers J M, Baker J V, & Blumberg B M (2003) Human herpesvirus 6 genome and antigen in acute multiple sclerosis lesions. J Infect Dis 187(9):1365-1376.
18. Berti R, Soldan S S, Akhyani N, McFarland H F, & Jacobson S (2000) Extended observations on the association of HHV-6 and multiple sclerosis. (Translated from eng) J Neurovirol 6 Suppl 2:S85-87 (in eng).
19. Daibata M, Taguchi T, Taguchi H, & Miyoshi I (1998) Integration of human herpesvirus 6 in a Burkitt's lymphoma cell line. Br J Haematol 102(5):1307-1313.
20. Hall C B, et al. (2008) Chromosomal integration of human herpesvirus 6 is the major mode of congenital human herpesvirus 6 infection. (Translated from eng) Pediatrics 122(3):513-520 (in eng).
21. Luppi M, et al. (1993) Three cases of human herpesvirus-6 latent infection: integration of viral genome in peripheral blood mononuclear cell DNA. (Translated from eng) J Med Virol 40(1):44-52 (in eng).
22. Nacheva E P, et al. (2008) Human herpesvirus 6 integrates within telomeric regions as evidenced by five different chromosomal sites. (Translated from eng) J Med Virol 80(11):1952-1958 (in eng).
23. Torelli G, et al. (1995) Targeted integration of human herpesvirus 6 in the p arm of chromosome 17 of human peripheral blood mononuclear cells in vivo. (Translated from eng) J Med Virol 46(3):178-188 (in eng).
24. Ward K N, et al. (2006) Human herpesvirus 6 chromosomal integration in immunocompetent patients results in high levels of viral DNA in blood, sera, and hair follicles. (Translated from eng) J Clin Microbiol 44(4):1571-1574 (in eng).
25. Gompels U A & Macaulay H A (1995) Characterization of human telomeric repeat sequences from human herpesvirus 6 and relationship to replication. (Translated from eng) The Journal of general virology 76 (Pt 2):451-458 (in eng).
26. Tanaka-Taya K, et al. (2004) Human herpesvirus 6 (HHV-6) is transmitted from parent to child in an integrated form and characterization of cases with chromosomally integrated HHV-6 DNA. (Translated from eng) J Med Virol 73(3):465-473 (in eng).
27. Gardella T, Medveczky P, Sairenji T, & Mulder C (1984) Detection of circular and linear herpesvirus DNA molecules in mammalian cells by gel electrophoresis. Journal of virology 50(1):248-254.
28. Britt-Compton B, et al. (2006) Structural stability and chromosome-specific telomere length is governed by cis-acting determinants in humans. (Translated from eng) Hum Mol Genet. 15(5):725-733 (in eng).
29. Graham F L, Smiley J, Russell W C, & Nairn R (1977) Characteristics of a human cell line transformed by DNA from human adenovirus type 5. (Translated from eng) The Journal of general virology 36(1):59-74 (in eng).
30. Ochman H, Gerber A S, & Hartl D L (1988) Genetic applications of an inverse polymerase chain reaction. (Translated from eng) Genetics 120(3):621-623 (in eng).
31. Delecluse H J & Hammerschmidt W (1993) Status of Marek's disease virus in established lymphoma cell lines: herpesvirus integration is common. (Translated from eng) Journal of virology 67(1):82-92 (in eng).
32. Delecluse H J, Schuller S, & Hammerschmidt W (1993) Latent Marek's disease virus can be activated from its chromosomally integrated state in herpesvirus-transformed lymphoma cells. (Translated from eng) EMBO J. 12(8):3277-3286 (in eng).
33. Leong H N, et al. (2007) The prevalence of chromosomally integrated human herpesvirus 6 genomes in the blood of UK blood donors. (Translated from eng) J Med Virol 79(1):45-51 (in eng).
34. Ward K N, Thiruchelvam A D, & Couto-Parada X (2005) Unexpected occasional persistence of high levels of HHV-6 DNA in sera: detection of variants A and B. (Translated from eng) J Med Virol 76(4):563-570 (in eng).
35. Baralle D (2001) Chromosomal aberrations, subtelomeric defects, and mental retardation. (Translated from eng) Lancet 358(9275):7-8 (in eng).
36. Luke B & Lingner J (2009) TERRA: telomeric repeat-containing RNA. (Translated from Eng) EMBO J. (in Eng).
37. Bandobashi K, et al. (1997) Human herpesvirus 6 (HHV-6)-positive Burkitt's lymphoma: establishment of a novel cell line infected with HHV-6. Blood 90(3):1200-1207.
38. Collins C M, Medveczky M M, Lund T, & Medveczky P G (2002) The terminal repeats and latency-associated nuclear antigen of herpesvirus saimiri are essential for episomal persistence of the viral genome. The Journal of general virology 83(Pt 9): 2269-2278.
39. Medveczky M M, et al. (1989) Herpesvirus saimiri strains from three DNA subgroups have different oncogenic potentials in New Zealand white rabbits. Journal of virology 63(9):3601-3611.
40. Neipel F, Ellinger K, & Fleckenstein B (1991) The unique region of the human herpesvirus 6 genome is essentially collinear with the UL segment of human cytomegalovirus. (Translated from eng) The Journal of general virology 72 (Pt 9): 2293-2297 (in eng).
41. Rieder M J, Taylor S L, Tobe V O, & Nickerson D A (1998) Automating the identification of DNA variations using quality-based fluorescence re-sequencing: analysis of the human mitochondrial genome. (Translated from eng) Nucleic Acids Res 26(4):967-973 (in eng).
42. Chang L K & Liu S T (2000) Activation of the BRLF1 promoter and lytic cycle of Epstein-Barr virus by histone acetylation. (Translated from eng) Nucleic Acids Res 28(20):3918-3925 (in eng).
43. Lu F, et al. (2003) Chromatin remodeling of the Kaposi's sarcoma-associated herpesvirus ORF50 promoter correlates with reactivation from latency. (Translated from eng) Journal of virology 77(21):11425-11435 (in eng).
44. Medveczky P G, Chang C-W, Oste C., and Mulder C (1987) Rapid vacuum driven transfer of DNA and RNA from gels to solid supports, Biotechniques 5 (3): 242-246.
Arbuckle J, Medveczky M M, Hadley S, Luegmayr A, Ablashi D, Lund T, De Meirleir K, Montoya J, Komaroff A L, Ambros P, and Medveczky P G. (2010) The Human Herpesvirus 6 DNA Genome Specifically Integrates in Telomeres of Human Chromosomes in Latency In Vivo and In Vitro. Proceedings of the National Academy of Sciences 107:5563-5568
Azzalin C M, Reichenbach P, Khoriauli L, Giulotto E, & Lingner J (2007) Telomeric repeat containing RNA and RNA surveillance factors at mammalian chromosome ends. Science (New York, N.Y. 318(5851):798-801.
Bandobashi K, et al. (1997) Human herpesvirus 6 (HHV-6)-positive Burkitt's lymphoma: establishment of a novel cell line infected with HHV-6. Blood 90(3):1200-1207.
Berti R, Soldan S S, Akhyani N, McFarland H F, & Jacobson S (2000) Extended observations on the association of HHV-6 and multiple sclerosis. J Neurovirol 6 Suppl 2:S85-87.
Borenstein & Frenkel N. (2009) Cloning human herpes virus 6A genome into bacterial artificial chromosomes and study of DNA replication intermediates PNAS 106: 19138-19143
Burgess Hickman A, Ronning D R, Perez Z N, Kotin R M, and Dyda F. (2002) The Nuclease Domain of Adeno-Associated Virus Rep Coordinates Replication Initiation Using Two Distinct DNA Recognition Interfaces. Molecular Cell 13: 403-414
Burns and Parrish, in Fields Virology (2007) 2437-2477
Caselli E, et al. (2006) Human herpesvirus 6 (HHV-6) U94/REP protein inhibits betaherpesvirus replication. Virology 346(2):402-414
Cermelli C, et al. (2003) High frequency of human herpesvirus 6 DNA in multiple sclerosis plaques isolated by laser microdissection. J Infect Dis 187(9):1377-1387.
Collins C M, Medveczky M M, Lund T, & Medveczky P G (2002) The terminal repeats and latency-associated nuclear antigen of herpesvirus saimiri are essential for episomal persistence of the viral genome. The Journal of general virology 83(Pt 9): 2269-2278.
Daibata M, Taguchi T, Taguchi H, & Miyoshi I (1998) Integration of human herpesvirus 6 in a Burkitt's lymphoma cell line. Br J Haematol 102(5):1307-1313
Daibata M, Taguchi T, Nemoto Y, Taguchi H, & Miyoshi I (1999) Inheritance of chromosomally integrated human herpesvirus 6 DNA. Blood 94(5):1545-1549.
Delecluse, H. J., Hilsendegen, T., Pich, D., Zeidler, R. & Hammerschmidt, W. (1998). Propagation and recovery of intact, infectious Epstein-Barr virus from prokaryotic to human cells. Proceedings of the National Academy of Sciences, USA 95, 8245-8250.
Dhepakson P, et al. (2002) Human herpesvirus-6 rep/U94 gene product has single-stranded DNA-binding activity. The Journal of general virology 83:847-854.
de Lange T (2005) Shelterin: the protein complex that shapes and safeguards human telomeres. Genes & development 19(18):2100-2110.
Deng Z, et al. (2002) Telomeric proteins regulate episomal maintenance of Epstein-Barr virus origin of plasmid replication. Mol Cell 9(3):493-503.
Deng Z, Norseen J, Wiedmer A, Riethman H, & Lieberman P M (2009) TERRA RNA binding to TRF2 facilitates heterochromatin formation and ORC recruitment at telomeres. Mol Cell 35(4):403-413.
Donati D, et al. (2003) Detection of human herpesvirus-6 in mesial temporal lobe epilepsy surgical brain resections. Neurology 61(10):1405-1411.
Gardella T, Medveczky P, Sairenji T, & Mulder C (1984) Detection of circular and linear herpesvirus DNA molecules in mammalian cells by gel electrophoresis. Journal of virology 50(1):248-254.
Geck, P., M. M. Medveczky, C.-S. Chou, A. Brown, and P. Medveczky. (1994). Herpesvirus saimiri small RNA and interleukin-4 mRNA AUUUA repeats compete for sequence-specific factors including a novel 70kd protein. J. Gen. Virol. 75:2293-2301
Goodman A D, Mock D J, Powers J M, Baker J V, & Blumberg B M (2003) Human herpesvirus 6 genome and antigen in acute multiple sclerosis lesions. J Infect Dis 187(9):1365-1376.
Graham F L, Smiley J, Russell W C, & Nairn R (1977) Characteristics of a human cell line transformed by DNA from human adenovirus type 5. The Journal of general virology 36(1):59-74.
Griffiths P D, Ait-Khaled M, Bearcroft C P, Clark D A, Quaglia A, Davies S E, Burroughs A K, Rolles K, Kidd M et al, (2000). Human Herpesviruses 6 and 7 as Potential Pathogens After Liver Transplant: Prospective Comparison With the Effect of Cytomegalovirus Transplantation. Journal of Medical Virology 59, 496-501
Hall C B, et al. (2008) Chromosomal integration of human herpesvirus 6 is the major mode of congenital human herpesvirus 6 infection. Pediatrics 122(3):513-520.
Jones C M, Dunn H G, Thomas E E, Cone R W, & Weber J M (1994) Acute encephalopathy and status epilepticus associated with human herpes virus 6 infection. Dev Med Child Neurol 36(7):646-650.
Kidd, I M; Clark, D A; Sabin, C A; Andrew, D; Hassan-Walker, A F; Sweny, P; Griffiths, P D; Emery, Vincent C. Prospective study of human Betaherpesviruses after renal transplantation: Association of Human Herpesvirus 7 and Cytomegalovirus Co-infection with Cytomegalovirus Disease and Increased Rejection. Transplantation. 69(11):2400-2404.
Leong H N, et al. (2007) The prevalence of chromosomally integrated human herpesvirus 6 genomes in the blood of UK blood donors. J Med Virol 79(1):45-51.
Luke B & Lingner J (2009) TERRA: telomeric repeat-containing RNA. EMBO J 28:2503-2510.
Luppi M, et al. (1993) Three cases of human herpesvirus-6 latent infection: integration of viral genome in peripheral blood mononuclear cell DNA. J Med Virol 40(1):44-52.
Lusso P, et al. (1989) Productive dual infection of human CD4+ T lymphocytes by HIV-1 and HHV-6. Nature 337(6205):370-373.
Lusso P, et al. (1991) Productive infection of CD4+ and CD8+ mature human T cell populations and clones by human herpesvirus 6. Transcriptional down-regulation of CD3. J Immunol 147(2):685-691.
Lusso P, et al. (2007) Human herpesvirus 6A accelerates AIDS progression in macaques. Proceedings of the National Academy of Sciences of the United States of America 104(12):5067-5072.
Marechal V, et al. (1999) Mapping EBNA-1 domains involved in binding to metaphase chromosomes. Journal of virology 73(5):4385-4392.
Martens U M, Zijlmans, J M, Poon, S S et al, (1998) Short telomeres on human chromosome 17p. Nat Genet. 18:76-80.
Mirandola, P., P. Menegazzi, S. Merighi, T. Ravaioli, E. Cassai, and D. DiLuca. 1998. Temporal mapping of transcripts in herpesvirus 6 variants. J. Virol. 72:3837-3844.
Mori, Y., P. Dhepakson, T. Shimamoto, K. Ueda, Y. Gomi, H. Tani, Y. Matsuura, and K. Yamanishi. 2000. Expression of human herpesvirus 6B rep within infected cells and binding of its gene product to the TATAbinding protein in vitro and in vivo. J. Virol. 74:6096-6104.
Muyrers J P, Zhang Y, Testa G, Stewart A F (1999). Rapid modification of bacterial artificial chromosomes by ET-recombination. Nucleic Acids Res. 27:1555-1557
Nacheva E P, et al. (2008) Human herpesvirus 6 integrates within telomeric regions as evidenced by five different chromosomal sites. J Med Virol 80(11):1952-1958.
Neipel F, Ellinger K, & Fleckenstein B (1991) The unique region of the human herpesvirus 6 genome is essentially collinear with the UL segment of human cytomegalovirus. The Journal of general virology 72:2293-2297.
Ng B L and Carter N P (2006) Factors Affecting Flow Karyotype Resolution. Wiley InterScience (www.interscience.wiley.com). DOI: 10.1002/cyto.a.20330
Owens R A, Weitzman M D, Kyostio S R, and Carter B J. (1993) Identification of a DNA-binding domain in the amino terminus of adeno-associated virus Rep proteins. J. Virol. 67: 997-1005
Raynaud C M, Sabatier L, Philipot O, Olaussen K A, & Soria J C (2008) Telomere length, telomeric proteins and genomic instability during the multistep carcinogenic process. Crit. Rev Oncol Hematol 66(2):99-117.
Takahashi K, et al. (1989) Predominant CD4 T-lymphocyte tropism of human herpesvirus 6-related virus. Journal of virology 63(7):3161-3163.
Tanaka-Taya K, et al. (2004) Human herpesvirus 6 (HHV-6) is transmitted from parent to child in an integrated form and characterization of cases with chromosomally integrated HHV-6 DNA. J Med Virol 73(3):465-473.
Thomson B J, Efstathiou S, & Honess R W (1991) Acquisition of the human adeno-associated virus type-2 rep gene by human herpesvirus type-6. Nature 351(6321):78-80.
Thomson B J, Weindler F W, Gray D, Schwaab V, & Heilbronn R (1994) Human herpesvirus 6 (HHV-6) is a helper virus for adeno-associated virus type 2 (AAV-2) and the AAV-2 rep gene homologue in HHV-6 can mediate AAV-2 DNA replication and regulate gene expression. Virology 204(1):304-311.
Torelli G, et al. (1995) Targeted integration of human herpesvirus 6 in the p arm of chromosome 17 of human peripheral blood mononuclear cells in vivo. J Med Virol 46(3):178-188.
Troy S B, Blackburn B G, Yeom K, Caulfield A K, Bhangoo M S, Montoya J G. Severe encephalomyelitis in an immunocompetent adult with chromosomally integrated human herpesvirus 6 and clinical response to treatment with foscarnet plus ganciclovir. Clin Infect Dis. 2008 47:e93-6.
Tsujimura H, Iseki T, Date Y, Watanabe J, Kumagai K, Kikuno K, Yonemitsu H, Saisho H. Human herpesvirus-6 encephalitis after bone marrow transplantation: magnetic resonance imaging could identify the involved sites of encephalitis. Eur J. Haematol. 61:284-5.
Ward K N, Thiruchelvam A D, & Couto-Parada X (2005) Unexpected occasional persistence of high levels of HHV-6 DNA in sera: detection of variants A and B. J Med Virol 76(4):563-570. Ward K N, et al. (2006) Human herpesvirus 6 chromosomal integration in immunocompetent patients results in high levels of viral DNA in blood, sera, and hair follicles. J Clin Microbiol 44(4):1571-1574.
Zhou J, Snyder A R, & Lieberman P M (2009) Epstein-Barr virus episome stability is coupled to a delay in replication timing. Journal of virology 83:2154-2162.

We claim:

1. A method for determining a subtelomere DNA sequence of a host, comprising:
a) providing a population of host cells that carry HHV-6 genomic DNA in host chromosomal DNA;
b) subjecting the host chromosomal DNA to inverse polymerase chain reaction (IPCR), thereby generating HHV-6-subtelomere DNA; and
c) determining the HHV-6-subtelomere DNA sequence.

2. The method according to claim 1, comprising, after step a), the step of: extracting chromosomal DNA from host cells.

3. The method according to claim 1, comprising determining the host chromosome that carries HHV-6-subtelomere DNA using fluorescence in situ hybridization (FISH), polymerase chain reaction (PCR), fluorescent labeling of HHV-6 DNA, Southern blot, or any combination thereof.

4. The method according to claim 3, wherein the host chromosome that carries HHV-6-subtelomere DNA is determined by fluorescence in situ hybridization (FISH) using HHV-6-specific DNA probes.

5. The method according to claim 3, wherein the host chromosome that carries HHV-6-subtelomere DNA is determined by Southern Blot using HHV-6-specific and/or chromosome-specific probes.

6. The method according to claim 1, wherein step b) comprises:
digesting the host chromosomal DNA using restriction endonuclease molecules, thereby generating host chromosomal DNA fragments comprising the HHV-6-subtelomere DNA; and
circularizing the fragments comprising the HHV-6-subtelomere DNA via intramolecular ligation.

7. The method according to claim 6, wherein step b) further comprises: diluting host chromosomal DNA fragments after restriction digestion.

8. The method according to claim 6, wherein the restriction endonuclease molecule is selected from the group consisting of MboI, Mme I, HaeIII, FspBI, Csp6I, NciI, NsiI, PovII, Esp3I, PstII, ClaI, BssHII, TaqI, RsaI, Sau3AI, HindIII, EcoRI, EcoRII, BamHI, NotI, SamI, AluI, KpnI, PstI, ScaI, and XbaI.

9. The method according to claim 8, wherein the restriction endonuclease molecule is MboI.

10. The method according to claim 1, wherein the host cells carry HHV-6A DNA, HHV-6B DNA, or both.

11. The method according to claim 10, wherein the host cells carry HHV-6A strain U1102 or HHV-6B Z29 strain.

12. The method according to claim 12, wherein the HHV-6-subtelomere DNA is generated using an IPCR primer that is complementary to at least 15 contiguous nucleotides of HHV-6 right end direct repeat (DR_{R}) DNA of any of U1-U100.

13. The method according to claim 1, wherein the IPCR primer is complementary to at least 15 contiguous nucleotides of any of HHV-6 U53, HHV-6-U54 and HHV-6-U94 DR_{R} DNA.

14. The method according to claim 12, wherein the IPCR primer comprises SEQ ID NO:1 or SEQ ID NO:2.

15. The method according to claim 1, wherein the sequence of the HHV-6-subtelomere DNA is determined using dideoxy sequencing reactions (Sanger sequencing), sequencing by synthesis using reversibly terminated labeled nucleotides, primer walking, shotgun sequencing, gel electrophoresis sequencing, multi-color fluorescence-based DNA techniques, sequencing by hybridization, DNA microarray, pyrosequencing, 454 sequencing, polony sequencing, SOLiD sequencing, or any combination thereof.

16. The method according to claim 1, wherein the chromosome of interest is of mammalian origin.

17. The method according to claim 1, wherein the host cells are isolated from an HHV-6-infected subject, and wherein the sequence of the HHV-6-subtelomere DNA indicates the host chromosomal telomeric site into which HHV-6 DNA is inserted.

18. A kit for determining the subtelomere DNA sequence of a chromosome of interest, comprising: an IPCR primer that is, or is complementary to, at least 15 contiguous nucleotides of one strand HHV-6 right end direct repeat (DR_{R}) sequence of any of U1-U100.

19. The kit according to claim 18, wherein the IPCR primer comprises SEQ ID NO:1 (IPCR-1) or SEQ ID NO:2 (IPCR-2).

20. The kit according to claim 18, further comprising one or more of materials selected from the group consisting of primer for host chromosomal-specific PCR, host chromosomal-specific hybridization probe, hybridization probe for identifying HHV-6 DNA, hybridization probe for identifying host telomere DNA, hybridization probe for identifying host subtelomere DNA, restriction enzyme molecules, DNA ligase, and Taq DNA polymerase.

21. The kit according to claim 18, comprising oligonucleotide molecules comprising any of SEQ ID NO:3-SEQ ID NO:40.


SEQUENCE LISTING (40 sequences)

SEQ ID NO: 1 - DNA, 28 bp, Artificial Sequence
  IPCR primer
gcacaacccacccatgtggtagtcgcgg

SEQ ID NO: 2 - DNA, 29 bp, Artificial Sequence
  IPCR primer
cgtgtgtacgcgtccgtggtagaaacgcg

SEQ ID NO: 3 - DNA, 20 bp, Artificial Sequence
  IPCR probe
cttacacttgccatgctagc

SEQ ID NO: 4 - DNA, 24 bp, Artificial Sequence
  telomere repeat probe
ttagggttagggttagggttaggg

SEQ ID NO: 5 - DNA, 26 bp, Artificial Sequence
  oligonucleotide primer/probe for HHV-6 DRR
catagatcgggactgcttgaaagcgc

SEQ ID NO: 6 - DNA, 25 bp, Artificial Sequence
  oligonucleotide primer/probe for HHV-6 DRL
ctttctcgctgtgcctcacgctgtc

SEQ ID NO: 7 - DNA, 24 bp, Artificial Sequence
  chromosomal-specific PCR primer
cagaccttggaggcacggccttcg

SEQ ID NO: 8 - DNA, 30 bp, Artificial Sequence
  chromosomal-specific PCR primer
aacatcgaatccacggattgctttgtgtac

SEQ ID NO: 9 - DNA, 24 bp, Artificial Sequence
  chromosomal-specific PCR primer
ctcatgtcctcggtctcttgcctc

SEQ ID NO: 10 - DNA, 29 bp, Artificial Sequence
  HHV-6 DRR probe
gcggagacacatagccttggcgggaagac

SEQ ID NO: 11 - DNA, 24 bp, Artificial Sequence
  chromosomal-specific probe
cccaagcaggttgagaggctgagg

SEQ ID NO: 12 - DNA, 20 bp, Artificial Sequence
  oligonucleotide primer/probe for HHV-6 DR-1
tgccgcttcaacttcacctt

SEQ ID NO: 13 - DNA, 20 bp, Artificial Sequence
  oligonucleotide primer/probe for HHV-6 DR-2
agatgtggagagaaacgcga

SEQ ID NO: 14 - DNA, 24 bp, Artificial Sequence
  oligonucleotide primer/probe for HHV-6 U94
tgttcttctgctaactcggacgca

SEQ ID NO: 15 - DNA, 24 bp, Artificial Sequence
  oligonucleotide primer/probe for HHV-6 U94
cagttccaatgggcgtggacaaat

SEQ ID NO: 16 - DNA, 24 bp, Artificial Sequence
  oligonucleotide primer/probe for HHV-6 U94
atccacgcgtcttccgtgactatt

SEQ ID NO: 17 - DNA, 24 bp, Artificial Sequence
  oligonucleotide primer/probe for HHV-6 U94
tgttcatgtcttccggcgaaaggt

SEQ ID NO: 18 - DNA, 20 bp, Artificial Sequence
  oligonucleotide primer/probe for mitochondrion DNA
tggacaaccagctatcacca

SEQ ID NO: 19 - DNA, 20 bp, Artificial Sequence
  oligonucleotide primer/probe for mitochondrion DNA
actaattaatcccctggccc

SEQ ID NO: 20 - DNA, 20 bp, Artificial Sequence
  oligonucleotide primer/probe for mitochondrion DNA
cctggggtgggttttgtatg

SEQ ID NO: 21 - DNA, 20 bp, Artificial Sequence
  oligonucleotide primer/probe for mitochondrion DNA
tttccccctctattgatccc

SEQ ID NO: 22 - DNA, 20 bp, Artificial Sequence
  oligonucleotide primer/probe for mitochondrion DNA
gtggccttggtatgtgcttt

SEQ ID NO: 23 - DNA, 20 bp, Artificial Sequence
  oligonucleotide primer/probe for mitochondrion DNA
tcactctcactgcccaagaa

SEQ ID NO: 24 - DNA, 20 bp, Artificial Sequence
  oligonucleotide primer/probe for mitochondrion DNA
ggagaatgggggataggtgt

SEQ ID NO: 25 - DNA, 20 bp, Artificial Sequence
  oligonucleotide primer/probe for mitochondrion DNA
tgaaacttcggctcactcct

SEQ ID NO: 26 - DNA, 20 bp, Artificial Sequence
  oligonucleotide primer/probe for mitochondrion DNA
agctttgggtgctaatggtg

SEQ ID NO: 27 - DNA, 30 bp, Artificial Sequence
  HHV-6A GFP primer U53L
ggcccgcggagtagttccgcgtcagaatgc

SEQ ID NO: 28 - DNA, 29 bp, Artificial Sequence
  HHV-6A GFP primer U53R
ggcggatcccattcgttttattgaacgcg

SEQ ID NO: 29 - DNA, 33 bp, Artificial Sequence
  HHV-6A GFP primer U54L
ggcaagcttgcaatggttaaaagttgttttttg

SEQ ID NO: 30 - DNA, 30 bp, Artificial Sequence
  HHV-6AGFP primer U54R
gcgccgcggctgcatacttgctaccggaac

SEQ ID NO: 31 - DNA, 30 bp, Artificial Sequence
  GFP-N2 vector primer (left)
cgcggatccattaatagtaatcaattacgg

SEQ ID NO: 32 - DNA, 31 bp, Artificial Sequence
  GFP-N2 vector primer (right)
cgcggatcccgccttaagatacattgatgag

SEQ ID NO: 33 - DNA, 24 bp, Artificial Sequence
  oligonucleotide primer/probe for C484 Stp-F
ctcagaacgcggcaacaaacttga

SEQ ID NO: 34 - DNA, 24 bp, Artificial Sequence
  oligonucleotide primer/probe for C484 Stp-R
tttcggcatacctggatcccatga

SEQ ID NO: 35 - DNA, 20 bp, Artificial Sequence
  oligonucleotide primer/probe for Cytochrome-C oxidase-F
ttcgccgaccgttgactatt

SEQ ID NO: 36 - DNA, 23 bp, Artificial Sequence
  oligonucleotide primer/probe for Cytochrome-C oxidase-R
aagattattacaaatgcatgggc

SEQ ID NO: 37 - DNA, 20 bp, Artificial Sequence
  oligonucleotide primer/probe for Beta actin-F
ctggaacggtgaaggtgaca

SEQ ID NO: 38 - DNA, 23 bp, Artificial Sequence
  oligonucleotide primer/probe for Beta actin-R
aagggacttcctgtaacaatgca

SEQ ID NO: 39 - DNA, 24 bp, Artificial Sequence
  U94 qPCR primer
acagccattcgatggttcccagaa

SEQ ID NO: 40 - DNA, 24 bp, Artificial Sequence
  U94 qPCR primer
aacgaactgggagacgtatgcgat